(19) United States
(12) Patent Application Publication
(10) Pub. No.: US 2011/0300528 A1
(43) Pub. Date: Dec. 8, 2011

(54) Methods for Bacteriophage Design

(76) Inventors: Sabah Abdel Amir Jassim, Clifton Estate Nottingham (GB); Ahmed Sahib Abdulamir, Sharjah (AE); Fatima Abu Bakar, Sharjah (AE)

(21) Appl. No.: 13/132,562

(22) PCT Filed: Dec. 3, 2009
(86) PCT No.: PCT/GB2009/051641

§ 371 Date: Aug. 17, 2011

(30) Foreign Application Priority Data
Dec. 3, 2008 (GB) 0822068.3

(51) Int. Cl. C12N 7/00; C12Q 1/70

(52) U.S. Cl. 435/5; 435/235.1

(57) ABSTRACT

Methods for designing and breeding phages are described. The methods include methods to design phages for previously resistant bacterial strains. The methods described do not use genetic manipulation techniques.

This invention relates to methods for designing and breeding viruses and to viruses bred by the method. More particularly, the present invention relates to the design and breeding of new bacteriophages, and to the bacteriophages obtained using the method.

Bacteriophages or “phages” represent the largest virus group (Ackermann and Dubow. 1987). Bacteriophages have been found which are may propagation in, and thus infect, most of the common groups of bacteria. Individual host ranges are usually narrow, a property which has been exploited in the epidemiological typing of bacteria, for example, coliphages (a type of T-phage) are bacteriophages that specifically infect Escherichia coli. Coliphages, with no specificity for serotype, have been used for a phage-typing scheme for E. coli O157:H7 (Ahmed et al., 1987). For rapid detection or identification of O157:H7, Ronner and Cliver (1990) isolated a coliphage specific for Escherichia coli O157:H7 from cattle manure samples. This coliphage, designated “AR1”, formed turbid pin-point (0.5 mm) plaques on cell lawns of 14 strains of O157:H7 (but not other E. coli) and Shigella dysenteriae. Although, coliphage AR1 forms plaques on cell lawns of Escherichia coli O157:H7, it does not produce visible cell lysis in broth culture (Ronner and Cliver 1990). This may suggest that AR1 is a temperate bacteriophage; whereas lysogenic cells of E. coli O157:H7 are immune to super-infection by the same phage. This explains their growth within the turbid pin-point (0.5 mm) plaque centres: the edge of each plaque is clear because most cells undergo lytic infection. Among the cells infected earlier, a few cells will have been lysogenized and will form visible microcolonies in the centre of the plaque. However, the appearance of a series of phage-resistant E. coli isolates, which showed a low efficiency of plating against bacteriophage PP01, led to an increase in the cell concentration in the culture (Mizoguchi et al 2003).

In the ecosystem both phages and bacteria are continually evolving, with bacteria becoming phage-resistant and phages evolving to maintain or improve infectivity of host bacteria (Levin et al., 1977; Lenski and Levin, 1985; Bohannan and Lenski, 1997; Mizoguchi et al., 2003). The evolutionary coexistence of phages with bacteria for millions of years granted a natural, very powerful and dynamic, source of antibacterial agents. The main problem which has faced scientists for phage-bacteria interaction is the development of resistance by bacteria against phages, coupled with the difficulty of obtaining sufficient numbers of phages specific for all, or most of the, strains of a bacterial species.

In the last decade many researchers have tried to find phages which are lethal to E. coli O157:H7 but not to other strains of E. coli. Phage PP01 was previously shown to efficiently and specifically lyse E. coli O157:H7 (Morita et al 2002; Mizoguchi et al., 2003), however, host-range mutants have also been reported (Mizoguchi et al., 2003). Tanji et al. (2005) found that a three-phage cocktail worked effectively in vitro (aerobically and anaerobically) but phages were not sufficiently optimized to free mice from E. coli infection during in vivo studies. This addresses the need to use specifically engineered and optimized lytic phages when in vivo use of phages is intended.

Phages are highly specific to one strain or few strains of a bacterial species and this specificity makes them unique in their antibacterial action. Therefore, phages have been considered as “smart” antibacterial agents rather than “dummy” ones like antibiotics. The ability of phages to recognise precisely their hosts, renders them favourable antibacterial agents especially because broad-spectrum antibiotics kill both the target bacteria and all the beneficial bacteria present in the farm or in the organism body (Merril et al., 2003). The advantages of using phages against bacteria as lytic agents are numerous. However, the inability to cover all strains of certain bacterial species along with the easy development of evolutionary resistance by bacteria against their phages, have made phage therapy or phage biocontrol unsuccessful (Vieu, 1975) and eventually led to replacement of phage therapy, in most countries, with antibiotic treatment (Barrow and Soothill, 1997).

The efficiency of the in vivo use of lytic phages relies mainly on how robust, rapid and specific an action phages are able to exert before the immune system of the body being treated will reduce them below the level of effectiveness. Therefore, it seems that the less robust, unoptimized, phages have less chance to succeed in abolishing in vivo bacterial infection than the robust optimized counterparts. Moreover, it seems that the successful in vitro challenge of the attacking phages against host bacteria might be limited by the unavailability of plenty of highly efficient and specific phages for challenging each pathogen successfully.

In this regard, Kudva et al (1999) have screened phages that bind to the O157 antigen and against phages that bind to common E. coli receptors, such as pili, fimbriae, flagella, LPS cores, and other outer membrane proteins. They found some O157 strains that were resistant to plaque formation by individual phages from which they concluded that the excess mid-range-molecular-weight LPS made by the plaque-resistant E. coli O157 strains may accumulate around cells in soft agar and influence phage attachment but diffuse from cells in liquid culture. Therefore, an appropriate length of the O-side chains and an optimal LPS concentration may be necessary to make the receptor available for phage interactions and/or to allow irreversible phage binding (Calendar, 1988).

On the other hand, phage-destroying LPS receptors are well known and in one example the tail spike protein has been fully characterised and functions in both adhesion to the host cell surface and in receptor destruction (Baxa et al., 1996; Steinbacher et al., 1997). Thus, movement of virions in the LPS layer before DNA injection may involve the release and rebinding of individual tail spikes rather than hydrolysis of the O-antigen (Baxa et al., 1996). This would suggest that effective infection might require normal LPS, thus, phage mutations seem to originate by alternation of LPS structure (Mizoguchi et al., 2003) giving a solid clue on the importance of LPS of the outer membrane in controlling the fate of phage attachment and the consequent phage infection of the host cell. Therefore, it can be inferred that the modification of LPS of the outer membrane of host bacteria may play a key role in controlling the phage-host interaction and consequently control phage infection.

In general phage host interactions are dependent on the binding of tail proteins to specific bacterial surface receptors (Pelczar et al., 1993). It seems that the development of a successful phage against E. coli must address the emergence of mutant strains, the phage binding and infection of E. coli not being controlled by a single receptor, and the many factors which contribute to phage resistance including alteration or loss of receptors for the target cell envelope (Heller, 1992; Barrow et al., 1998; Biswas et al., 2002; Mizoguchi et al., 2003). Thus, the efficient use of phages to control E. coli infections may require isolation of mutant E. coli-specific phages that can adsorb to hosts that make shorter O-side chains (Kudva et al., 1999) This could suggest that phages need to be redesigned, namely, bred and “retailored” on the host cells in order to gain newly bred sub-strains of phages which are able to infect previously resistant bacteria and to play an important role in the future phages breeding applications, including the pre-harvest pathogen reduction strategies.

Phage breeding can be defined as the procedures pursued in modifying the physical, kinetic and biological characteristics of bacteriophages, leading to the formation of a newly bred strain or sub-strain. Phage breeding can loosely be categorized into two types; non-genetic and genetic breeding.

By “non-genetic”, as used herein is intended a method whereby the modifications to the phage are induced using culture methodology and reproduction and enhanced or forced natural selection techniques rather than by direct manipulation of the viral genome (“genetic breeding”) by manual deletion/insertion/replacement of nucleic acid sequences which specifically alter the genome of the phage in a pre-selected or well defined manner. The non-genetic method of the invention is environmentally-driven and so mimics natural selection or evolution of the phage by reproducing vast numbers of mixed populations of wild-type phages.

The selection of virus progeny using viricidal agent separation or neutralisation of extracellular virus once the more efficient virus particles have attached to and/or infected the target cell is known (Jassim et al. 1995; WO 95/23848).

Genetic virus design/breeding which is a genetic manipulation of the virus genome has been reported (Duenas and Borrebaeck, 1995; Rieder et al., 1996; O'Sullivan et al., 1998). However, to date, the genetic breeding of bacteriophages is still in its beginning stages with no rewarding results so far primarily due to the inability to manipulate phage genetics (Barrow and Soothill, 1997; Alisky et al., 1998).

However, the art is silent on a non-genetic method of virus breeding, in terms of modifying host-specificity such that previously phage-resistant bacterial strains become susceptible to phage infection.

It is therefore an object of the present invention to provide a non-genetic, or environmentally-driven, method for breeding bacteriophages which infect previously resistant bacterial strains.

The object of the horizontal breeding techniques of the present invention is to breed new phage progenies by chemical/physical re-adaptation of their host specificities to become lytic to new host bacteria that previously were resistant to the parent phage. By this technique, it is possible to design new phage specificities, non-genetically, toward target host bacteria and convert these phage-negative host cells to phage-positive host cells. This was achieved by an innovative standardization methodology to suit the nature of bacteria in general and E. coli in particular. This methodology will serve as a template breed phages against host resistance.

Several chemical substances were used in controlled physical conditions to supplement cultures of target phage-negative E. coli bacteria mixed non-specific coliphages to physically/chemically readapt the cell wall and the outer membrane of the target host cells to turn phage-sensitive. The mixture of chemical substances at certain physical conditions was called the “breeding solution”. The breeding solution is designed to modify the outer membrane permeability, specificity, receptors exposure, and membrane texture, as well as to change the conformation of the exposed moieties of LPS and teichoic acid, or to expose some hidden moieties in a non-specific way allowing new chances for the attacking phages to find new spots of recognition. Once the tail fibres and the baseplate of the attacking phage attach quite firmly to the newly recognized moieties, the insertion of their nucleic acids will be triggered immediately to pass through the cell wall into the interior of the bacterial cell and start the lytic infection process. The hypothesis of the current methodology of the invented horizontal breeding is to create an artificially-designed microenvironment, in the breeding solution, for the attacking phages to unusually succeed in infecting a naturally resistant strain of bacteria and produce altered phage progenies that acquired the specificity of the new host. Since most of E. coli bacteria are infected already with many lysogenic inert prophages, it is hypothesized that there is a possibility of some kind of genetic or epigenetic interaction between the artificially-driven lytic phages and the prophages, remnants of prophages, or the host DNA itself inside the bacterial cell. It was speculated that this might lead to a gaining of new specificity genes for the phage tail fibres to recognize the new moieties on the outer membrane of the target E. coli bacteria.

A large number of horizontal non-genetic breeding protocols were carried out. The design of these protocols was dependent mainly on the concept of modifying, changing, and partially tearing the cell wall of the host bacteria to become artificially susceptible to phage infection. Therefore, many pilot experiments underwent many changing protocols, different concentrations of the reagents used, different physical modifications different incubation time periods, and different chemical combinations used. After a series of time-consuming experiments on a high number of protocols, it was found that 3 protocols showed pretty good success and 1 protocol gave only very mild success.

Accordingly, the present invention provides a method of modifying phage-host specificity, the method comprising incubating phages in a medium comprising of one or more of a chelating agent, a detergent, a surfactant, an enzyme, a lantibiotic, an antibiotic and an agent which destroys cell walls.

Preferably, the invention provides a method of selectively breeding bacteriophages, in which the method comprises the steps of:—
(a) obtaining large amounts of wild-type phages from at least one natural source by incubating the phages with bacterial hosts to obtain large numbers of phages,
(b) removing bacterial host cells, to obtain a suspension of phages,
(c) plating the suspension of phages from step (b) on a lawn of bacterial host cells,
(d) assessing phage plaques to identify areas of highest phage activity,
(e) isolating the areas of highest phage activity and isolating phages therefrom,
(f) culturing the phages isolated in step (e) together with their host bacteria,
(g) adding a viricidal mixture to the culture media of step (f) to remove free phages from the culture medium,
(h) plating the viricidally-treated culture medium from step (g) onto a host bacterial lawn and identify plaques,
(i) removing the plaques showing most virulent phage activity from the plate and isolate the phages therefrom,
(j) incubating the phages obtained in step (i) in a medium comprising of one or more of a chelating agent, detergent/surfactant, enzyme, lantibiotic, antibiotics and an agent which destroys cell walls,
(k) isolating the bacteriophages of step (j) and incubating them in a growth medium,
(l) assessing the infectivity of the bacteriophages of step (k) and culturing those whose specificity has been modified,
(m) storing the bacteriophages cultured in step (l).

In a preferred embodiment of the method, the bacteriophages are obtained from one or more of animal or bird faeces, animal or bird litter, sewage, soil, or farmyard slurry. More preferably, the bacteriophages are obtained from one or a mixture of camel faeces, quellae litters, pigeons litters, chicken litters, sheep faeces, goat faeces, cattle faeces, cattle manure, cattle farm sewage, farm soil, water sanitization, regular swimming pools, fish ponds, lakes, oceans, water features, and hospitals.

Preferably, the bacteriophages are specific for one or more of Escherichia coli, Enterbacteriacea spp., Salmonella typhimurium, Pseudomonas aeruginosa, Bacterioides gingivalis, Actinobacillus actinomycetescomitans, Klebsiella pneumoniae or Gram positive bacteria such as Staphylococcus aureus including MRSA, Streptococcus mutans, Listeria monocytogenes, Streptococcus agalactiae, Coryneform bacteria, Mycobacterium tuberculosis, some strains of Salmonella spp., Campylobacter jejuni, water-borne Vibrio cholerae, or Helicobacter pylori. Most preferably, the bacteriophage infect one or more of Escherichia coli, Klebsiella pneumoniae, and Mycobacterium smegmatis.

Preferably, step (h) is carried out in the same medium as steps (a) to (f).

Preferably, the medium of step (j) comprises one or more of EDTA, lysozyme, Nisin A and Tween® 20. In the most preferred embodiment, all of EDTA, lysozyme, Nisin A and Tween® 20 are present in the medium.

In step (a) it is preferred that the phages are incubated in a broth culture medium. The broth may be a selective broth or simply one which promotes or is directed to the culture and growth of the host organism. For example, a tryptone broth is useful in the cultivation and breeding of enterobacteria. In the most preferred embodiment, especially where E. coli and coliphages are being grown, Luria broth is used. Optionally, the Luria broth may be supplemented with 10 g/l NaCl as in LB-Miller broth.

The host bacteria co-incubated with the phages in step (a) are the bacteria for which a phage is being sought. More than one host strain may be used in the same culture broth. The bacteria may be commercially available strains, clinical isolates, mixtures of strains, crude infected material, or the like. Optionally, the strain may be purified.

In step (b) the bacterial hosts may be removed by conventional methods such as centrifugation, addition of antibacterial compounds, lysis, or combinations thereof. Preferably, the bacteria are removed using a combination of centrifugation and chloroform digestion.

The present inventors noted that adding 1:1 volume chloroform to the supernatant caused 2-3 logs decrease of the phages present in the sample. Taking into account that concentration of some phages might be not more than 3 logs, it was decided that 1:1 volume of chloroform could abolish the chance to discover the low concentration phages within the crude sample mixture. Therefore, it was advantageous to use a 1:10 volume of chloroform: crude solution.

The phages obtained in this way (step (c)) are plated on a lawn of host bacteria which are preferably grown on a solidified version of the same broth as used in step (a). Therefore, in the most preferred example, the host bacterial lawn is formed on a Luria Broth agar plate, supplemented as above.

Areas of high phage activity are identifiable by the nature of the plaque or lysis zone formed in the lawn by the phages. The plaque morphology and growth are assessed and recorded in order to isolate the most virulent phages.

Preferably, the plaques in steps (d) and (h) are assessed for diameter, shape, depth, margin of cut, clarity. The plaques may also be used to assess the biokinetic criteria of the phages, as will be described in more detail below. The biokinetic criteria may be assessed by measuring the number of phages before and after burst of the phages. Additionally, the biokinetics may be assessed using data regarding, inter alia the ratio of infectivity, the burst time, and the burst size.

Preferably, in steps (h) and/or (i) the plaques are identified and then further selected by their biokinetic profile.

Optimal phage selection may be obtained by repeating steps (a) to (e) or steps (f) to (i) or both.

The phages obtained in step (d) are then isolated from the plaque and cultured as above. Steps (b) to (e) may be carried out more than once. It has been found preferable to repeat this step in order to optimize the phages obtained for virulence and other biokinetic properties. The phage amplification assay (Stewart et al 1998) has not been used here to avoid the loss of the amplified phages by their adherence to the surface of the used test tubes. Therefore the present inventors have designed a unique methodology of biokinetic measurement by using a single tube harbouring the whole series of biokinetic reactions without ever changing the tube which is called the “master tube”. This crucial innovation was found to be necessary to troubleshoot the setbacks of the traditional biokinetic assays which lack the desired preciseness as many phages are mistakenly overlooked and removed with changing each reaction tube.

The preferred method for assessing the biokinetics of the phage was as follows. A sample of phage is added to a bacterial culture and incubated before exposure to a viricidal agent, in the incubation vessel. After exposure to the viricidal agent, a surfactant is added to the mixture in the incubation vessel and further incubated. Culture broth is added to the incubation vessel. Samples are removed from the incubation vessel and added to fresh culture medium prior to plating on a bacterial lawn and assessment of plaque morphology. Optionally, a serial dilution may be preformed prior to plating.

Preferably, the phage and bacteria are co-incubated prior to the addition of the viricidal agent for a period less than an hour, more preferably of up to 20 minutes and ideally for a time of between 2 and 20 minutes.

In a preferred embodiment, the phage and bacteria are exposed to the viricidal agent for a period less than an hour, more preferably of up to 20 minutes and ideally for a time of up to 10 minutes.

Preferably, surfactant is added to the incubation vessel containing the phage, bacteria and viricide for a period of less than a minute, more preferably of up to 30 seconds and ideally for a time of up to 10 seconds.

To remove the unwanted phages from the culture broth, a viricidal agent is applied. Virulent phages or phages with improved biokinetic properties which have infected a host bacterial cell are not killed by the application of the viricide, but unbound and non-internalised phages in the broth will be. In the preferred embodiment of the invention, the viricide comprises pomegranate rind extract, iron salts and a detergent or surfactant. For biokinetic determination it is also preferred that the viricide comprises pomegranate rind extract.

The pomegranate is the fruit of a deciduous shrub native to Southwest Asia and has been cultivated in the Caucasus since ancient times. It is widely cultivated throughout Armenia, Azerbaijan, Iran, Turkey, Afghanistan, Pakistan, North India, the drier parts of southeast Asia, Peninsular Malaysia, the East Indies, and tropical Africa and was introduced into Latin America and California by Spanish settlers in 1769, where the pomegranate is now cultivated in parts of California and Arizona for juice production. In the Indian subcontinent's ancient Ayurveda system of medicine, the pomegranate has extensively been used as a source of traditional remedies for thousands of years. For example, the rind of the fruit and the bark of the pomegranate tree is used as a traditional remedy against diarrhoea, dysentery and intestinal parasites while the seeds and juice are considered a tonic for the heart and throat. The astringent qualities of the flower juice, rind and tree bark are considered valuable for a variety of purposes such as stopping nose bleeds and gum bleeds, toning skin, (after blending with mustard oil) firming-up sagging breasts and treating haemorrhoids. Pomegranate juice (of specific fruit strains) is also used as eye drops as it is believed to slow the development of cataracts.

The first step for the phage bio-kinetics is to prepare a potent antiviral (anti-phage) substance capable of neutralizing/destroying the phages without harming the target cells. Hence, infected bacterial hosts will act as a shelter for the phages to escape killing by the antiviral substance, this can partly be achieved as described by WO/1995/023848. Note, the antiviral substance reported in the patent WO/1995/023848 has never been tested for E. coli phages and nor on E. coli cells.

From the preliminary experiments, it was shown that the antiviral agent from WO/1995/023848, when used against isolated E. coli phages was active only for approximately 15 minutes after the preparation. Furthermore, the viricidal assay results obtained were not completely reliable as the neutralizing step (Tween 80) was currently found not efficient enough to completely inactivate the viricidal agent after an exposure contact time of 2, 5 and 10 min. However, since the fundamental objective of bio-kinetics assay is to measure precisely the contact time, the burst size, and the burst time of the tested phages, therefore it was necessary to apply a sharp cut and completely reliable neutralizing step for the antiviral substance. In this study, it was advantageously found that a new neutralizing solution proved to be 100% effective which is a combination of a specific concentration of Tween 20, instead of Tween 80, with Luria broth that gave the optimal neutralization effect ever done. This combination of LB and Tween 20 at certain ratio proved to act uniquely that neither Tween 20 nor LB could do the same neutralization job alone.

The pomegranate rind extract (PRE) is preferably made as follows. Pomegranate rind is blended in distilled water (25% w/v) and boiled for 10 minutes before centrifuging at 20 000×g for 30 minutes at 4° C. and autoclaved at 121° C. for 15 minutes and allowed to cool. The extract is further purified by membrane ultra-filtration at a molecular weight cut-off of 10 000 Da and stored at −20° C. until used. A preparation of 13% PRE is generally used which is prepared by diluting 1.3 ml of PRE (25% w/v) with 8.7 ml of buffer.

The iron salt is preferably ferrous sulphate (FeSO₄) although other ferrous salts may be used. The detergent/surfactant is preferably a polysorbate surfactant such as Tween®. For biokinetic determination it is also preferred that the detergent/surfactant is a polysorbate surfactant such as Tween®. Most preferably, the detergent/surfactant is Tween® 20. Preferably, the PRE is present at a concentration of between 3.25 and 7.5%, the ferrous sulphate at a concentration of between 0.01 and 0.04%, and the Tween® 20 at a concentration of between 0.1 and 10%.

In the ideal embodiment the viricidal agent is composed of 3.25% pomegranate rind extract (PRE) and 0.01% ferrous sulphate whilst the detergent/surfactant is 1.6% Tween® 20.

The phage specificity may be modified to infect previously resistant strains of the same bacteria, to infect different strains of bacteria, or to infect a different species of bacteria.

In a second aspect of the invention, the method of altering phage specificity may be carried out independently of the phage breeding method.

In a third aspect of the invention, the phages produced by the methods of the present invention are usable in various antibacterial applications. For example, phage biocontrol for pathogenic E. coli in livestock at the pre-harvest stages of the production process of plain meat, ground meat, and poultry, prophylactic animal feed with coliphage in drinking water or food, for example using absorbable vegetable capsules filled with phage cocktail, bioprocessing of the machinery and tools used in food industry plants, restaurants, hospitals, in humans postinfection, in animals preslaughter, in foods postharvest, food preservative, food additive slaughter houses as E. coli biofilms might form and lead to serious persistent sources of infection, prevent and/or eliminate the biofilms of E. coli formed on the surface of urinary catheters, in phage-based rapid diagnostic testing, or in phage therapy for E. coli infections either by topical or systematic routes of administration in which the rapid bacterial lysis of the specific action phages can exerted before the immune system of the host body can be developed.

Embodiments of the invention will now be described by way of example only, with reference to and as illustrated by the following Examples.

Materials and Methods

### Media

Luria broth (LB): tryptone 10 g l⁻¹ (HiMedia, Mumbai, India), yeast extract 5 g l⁻¹ (HiMedia, Mumbai, India), and sodium chloride 10 g l⁻¹ (HiMedia, Mumbai, India) at pH 7.2 were used in all the protocols. L-agar (LA), consisted of the above with the addition of 14 g l⁻¹ agar (HiMedia, Mumbai, India) was used for culture maintenance. Bacterial dilutions from 18 h LB cultures grown at 37° C. were carried out in phosphate buffered saline (PBS, Oxoid, UK). For plaque assay, the ‘soft layer agar’ used was LB prepared in Lambda-buffer [6 mmol l⁻¹ Tris pH 7.2, 10 mmol l⁻¹ Mg(SO4)2.7H2O, 50 μg ml-1 gelatin (Oxoid, UK)], was supplemented with 4 g l⁻¹ agarbacteriology No. 1 (HiMedia, Mumbai, India).

### Phage Vertical Breeding

The first phase of the vertical breeding was a new technique to hunt as many as specific phages in a very short time. One hundred and twenty one phages were hunted and isolated from wild. Then a series of phage optimization steps have been implemented on the isolated wild phages. This kind of optimizations is called vertical breeding as it has bred the same phage to a better sub-strain without changing the host range.

### Optimization of Phage Isolation

### Phage Isolation and Propagation

A series of optimization steps have been introduced in order to augment the efficacy and art of phage hunting/isolation techniques. The optimization manoeuvres were taken into account:
i) The crude phage samples collection was diversified in a way that 1 g of 10 different crude samples of camel faeces, quellae litters, pigeons litters, chicken litters, sheep faeces, goat faeces, cattle faeces, cattle manure, cattle farms sewage and farms soil were mixed and called “crude mixture”.
ii) Samples of crude mixture representing 10 g were placed in 100 ml Erlenmeyer flask with cotton-plugged. Then 80 ml of LB was added and the mixture was inoculated with a total of 10 ml of ten 18 h cultures E. coli (1 ml each) clinical isolates or the representative NTCC and ATCC E. coli strains.
iii) After 18 h standing incubation at 37° C., sample of 10 ml was dispensed into a sterile 15-mL plastic culture tubes.
iv) After centrifugation at 5000×g for 5 min at room temperature the supernatant transferred into 1.5 ml sterile microcentrifuge. Then 1:10 chloroform to lysate ratio was added with gentle shaking for 5 minutes at room temperature in order to lyse the bacterial cells followed by further 3 min incubation in crushed ice the mixture has centrifuged for at 5000×g for 15 min at room temperature and the supernatant transferred into a 1.5 ml sterile microcentrifuge tubes which became now the isolated phages mixture.
v) Then, the produced mixture of the isolated phages was propagated on the desired target bacteria lawn as it is earlier mentioned in the procedure of the phage spot lysis test.

Hence, the present inventors have obtained an ever increasing number of crude mixture-purified and isolated multi-phages covering the high number of the mixed clinical E. coli isolates and the reference strains. Accordingly, this will accelerate the identification and hunting of new phages as large number of target host bacteria and potential phage crude samples are mixed in one tube saving time and effort as well as maximising the possibilities of phage hunting.

### Production of the Transient Phage Stock

The mixture of the isolated phages from iv) above was propagated on each target bacterial lawn as mentioned earlier for the phage spot lysis test: phages were propagated from their own lysis zones on the bacterial lawns. Lysis zones, if any, were cut by a sterile scalpel and plunged into 300 μl of Lambda buffer in 1.5 ml sterile microcentrifuge tubes for 20 minutes with intermittent gentle shaking. 1:10 chloroform to lysate ratio was added with gentle shaking for 5 minutes at room temperature in order to elute the phages from the agar and to lyse the bacterial cells. After further 3 min incubation in crushed ice the mixture centrifuged at 5000×g for 15 min at room temperature and the supernatant transferred in a 1.5 ml sterile microcentrifuge tubes.

The transient phage stock solution should contain approximately 10⁵ to 10⁷ PFU ml⁻¹.

### Optimization of the Phage Lytic Characteristics

Plaque-Based Optimization

The isolates of wild lytic phages from the transient stocks were propagated with the corresponding host clinical E. coli isolates and the representative NTCC and ATCC reference E. coli strains using the plate method as follows: Ten folds serial dilutions (10⁻¹ to 10⁻⁶) were made with Lambda buffer for the phage stock solutions by taking 100 μl of the phage solution into 900 μl of lambda buffer. Transfer of 100 μl of each dilution for each phage stock solution into 15 ml volume sterile plastic container contain 100 μl of 10⁹ CFU ml⁻¹ of 18 h LB culture of targeted bacteria and incubate at 37° C. After 10 min incubation, the added 2.5 ml of top layer agar cooled to 45° C. and poured over L-agar plates. Plates were incubated overnight at 37° C. and plaque morphology, growth characteristics were recorded according to the following parameters:

a) Diameter (mm) of the plaque.

b) Shape of the plaque.

c) Depth of the plaque

d) Margin cut.

e) Clarity or turbidity of the plaque.

f) Plaque visible time.

By conducting a thorough examination of the formed plaques, it was found that only very few out of tens or hundreds of plaques per plate show larger diameters and clearer lysis than the average. The difference in plaque size has long been underestimated and overlooked as it is very slight and hard to notice. The slightly larger plaques proved to be an excellent indicator for the optimization of the phages lytic characteristics by using the vertical breeding. Accordingly, the best 3-5 well-defined, clear, and largest plaques were selected at each run and used according to the above phages purification and propagation program. This has been repeated for 8-10 runs in order to magnify the outcome of the biased selection of the large and clear plaques thus obtaining the ever-largest and the ever-clearest 3-5 plaques, reflecting the best yet possible enhancement of the lytic characteristics of the bred phages.

### Biokinetic-Based Optimization

This optional step was carried out on the phages recovered from the 3-5 optimized plaques that resulted from the plaque-based optimization technique. This step was used to choose the phage set which shows the highest biokinetic values given that remarkable differences in the biokinetic values were seen among the tested phage sets which might have been overlooked by previous plaque-based optimization techniques.

One of the main advantages of the current biokinetic tests are that the accuracy of the assay which relies only on a single tube known as “master tube” which is wholly different from all previous biokinetics assays. This novel approach allows estimating phage burst size, burst time, contact time, ratio of infectivity of the isolated or bred phage much more accurately.

The viricidal agent used in this protocol is composed of 400 μl of 3.25% pomegranate rind extract (PRE) and 600 μl of 0.01% FeSO4 and is active for 45 min after preparation, whilst the neutralizer agent is composed from 8% Tween 20 with contact time of 5-10 sec followed by the addition of LB up to 1 ml total volume.

### Design of the Biokinetic Assay:

The above viricidal agent alongside with the neutralizing materials proved to be perfect phage destroying and neutralizing substances respectively without harming the target cell “E. coli”.

The innovative single master tube biokinetic protocol was conducted as follows:

10 μl (10¹² PFU ml⁻¹) of phage+10 μl of bacteria (10⁵ CFU ml⁻¹)→contact time 2, 5, 10, 15, and 20 min→100 μl viricidal agent, exposure time 10 min→200 μl of 8% Tween 20 contact time 5-10 sec→680 μl of LB were added to make it up to 1 ml→Transfer 10 μl in micro-centrifuge tube containing 900 μl Lambda buffer, so 10-fold serial dilutions were prepared. From each dilution, 10 μl were spotted on the appropriate bacterial lawn of LA at timely intervals; zero, 10, 20, 30, and 40 minutes past the neutralization step to recover the formed plaques before and after the burst of the new phage progenies. The plates were then incubated at 37° C. for 18 h.

### Interpretation of the Biokinetic Assay:

The interpretation of the results was classified into two eras; the pre-burst era and post-burst-era. At the pre-burst era, the number of the plaque forming units (PFU) or plaques is equal to or less than the number of the bacteria used in the test for the given dilution. In this era, each plaque was formed by lysis of one bacterial cell releasing high number of phage progenies in situ leading to formation of a plaque. That means each bacterial cell sheltered certain number of replicating phages which will then form a plaque.

The time after the burst time is considered as post-burst era. In this era, each plaque represents a new phage progeny which was released in the master tube before spotting onto the lawn. Hence in this assay plaques represent two meanings according to the pre- or post-era of the assay.

Therefore the interpretation will be as follows:

Phage binding time (PBT): The time for the encounter between bacterial hosts and their specific phages that gives the highest number of phage particles at the pre-burst era or yields the highest infective ratio.

Infective ratio (IR): it is the ratio between the number of phage particles at the pre-burst era and the number of the bacterial hosts used in the assay. IR=No. of phage particles in the pre-burst era at a given dilution/No. of the bacterial hosts used in the assay at the same given dilution. The closer number of plaques in the pre-burst era to the bacterial titre used, the higher the IR.

Burst time (BT): it is the time measured before a sharp increase was observed in the number of the formed phage particles more than the number of the bacteria used for the given dilution. In other words, it is the time when the new phage progenies became responsible for the formation of plaques rather than their infected host cells.

Burst size (BS): The number of new phage progenies per one bacterial cell host. BS=No. of phage particles at the post-burst era/No. of the phage particles at the pre-burst era for the given dilution.

### Formation of the Optimized Definitive Phage Stocks

The elite phages were propagated from the best of the vertically-bred plaques using the above described plaque-based and/or biokinetic-based novel optimization methods. Lambda buffer was used as the recovery medium. Definitive phage stocks or the optimized phage stocks were developed on their appropriate host strains by a plate lysis procedure essentially equivalent to growing bacteriophage Lambda-derived vectors (Ausubel et al. 1991). Briefly, preparation of large volume of the optimized phages was conducted by using the soft layer plaque technique and as follows: An aliquot (100 μl) of the phage sample (10-fold serially diluted with lambda-buffer) was mixed with 100 μl of an overnight LB culture of E. coli clinical isolates and/or representative E. coli reference strains in a sterile Eppendorf micro-centrifuge tube (polypropylene; 1.5 ml; Sarstedt) and incubated for 10 min at 37° C. to facilitate attachment of the phage to the host cells. The mixture was transferred from the Eppendorf micro-centrifuge tube to a 5 ml Bijou bottle and then 2.3 ml of ‘soft agar’ was added (LB prepared in lambda-buffer and supplemented with 0.4% w/v agar bacteriology No. 1 Oxoid which had been melted and cooled to 40° C. in a water bath). The contents of each bottle were then well mixed by swirling, poured over the surface of a plate of LA and allowed to set for 15 min at room temperature. The plates were incubated for 18 h at 37° C., and a plate showing almost confluent plaques was used to prepare a concentrated phage suspension by overlaying with 5 ml of lambda-buffer [titre 10¹² plaque-forming units per ml (PFU)]. The final purification process used 1:10 chloroform to lysate ratio to separate the bacteriophage from the bacterial cells. The phage stocks were maintained in lambda-buffer at 4° C.

### Horizontal Breeding (Chemical/Physical Re-Adaptation of the Phage-Host Specificity)

### EXAMPLE 1

### Tween-20-Based Breeding

Tween 20 (Merck, Germany), also known as polysorbate 20, was used in the standardisation trials. Tween 20 is considered an active substance against proteins and lipids but, unlike ethylene diamine tetraacetic acid (EDTA), it lacks a potent chelating potential for cations which are considered one of the main pillars of the cell membrane solidity. Different concentrations of Tween 20 were tested in the horizontal breeding technique it was found that 1.6% of Tween 20 was the optimal concentration achieved and as follows:

Transfer 200 μl of 8% Tween 20 to 800 μl of an 18 h LB culture of E. coli clinical isolates and/or the representative NTCC and ATCC reference E. coli strains in a sterile Eppendorf micro-centrifuge tube (polypropylene; 1.5 ml; Sarstedt). Therefore the final concentration of Tween 20 is 1.6%. Then 200 μl of total 20 isolates of wild coliphages were added in a quantity of 10 μl (10¹² PFU ml⁻¹) per a phage and incubated at 37° C. After 18 h, 100 μl of 10 strengths of LB were added followed by the addition of 10 μl of each of the used 20 phage stocks and a loopful of 18 h LA culture of the same target bacteria was added too. This was repeated for 10 days progressively.

At day 10, thin bacterial lawns of the same target bacteria were prepared and 10 μl of the Tween 20-treated phages were added on bacterial lawns and then incubated at 37° C. and plaques were observed after 6 h and 18 h. The detection of phage presence was based on visual appearance of lysis zone at the site of 10 μl solution added onto the surface of the lawn. Positive results were expressed by either clear or semi-clear (turbid) lysis zone while negative results were expressed by the absence of such lysis zones.

Results were shown as very mildly successful. Lysis spots of the harvested Tween 20-treated phages revealed very slight progress by contrast of negative result from untreated phage.

### EXAMPLE 2

### EDTA-Tris Buffer-Based Breeding

EDTA (ethylene diamine tetraacetic acid) is believed to act strongly on the outer cell membrane of E. coli, increasing the permeability of the membrane. This is one of the necessary requirements for successful cross linking of EDTA with phage and bacteria.

Since EDTA could be lethal to the bacteria at certain levels (Loretta, 1965) different concentrations of EDTA were prepared and a sub-lethal concentration of EDTA on the tested E. coli bacteria was used. From a series of lengthy standardization trial and error experiments, it was found that supplementing of Tris-HCl buffer at a concentration of 12 mM with 1 mM EDTA, the bacterial survival rate after two hours in the solution was not affected by the EDTA, therefore, this preparation was considered to be tested and used for the phage breeding assays as follows:

Transfer 1 ml of 8 h LB cultures of E. coli clinical isolates and the clinical isolates or the representative reference E. coli strains into 1.5 ml sterile microcentrifuge tubes and centrifuged for 10 min at 5000×g at room temperature. The supernatant was discarded and pellets were resuspended with 1 ml of 12 mM Tris-HCl (Sigma, USA) buffer (pH 8) and 1 mM EDTA (Merck, Germany) solution then incubate for 10 min at room temperature. The mixture was centrifuged for 10 min at 5000×g at room temperature. The supernatant was discarded and the pellets were resuspended with 1 ml of LB supplemented with 200 μl of 20 different vertically bred coliphages, each phage represented in 10 μl 10¹² PFU ml⁻¹ and incubated at 37° C. After 18 h, the mixture of 20 phages and the pre-treated Tris-EDTA bacteria was centrifuged at 5000×g at room temperature for 10 minutes and the resulting bacterial pellets was discarded and the supernatant added to a freshly treated Tris-EDTA bacterial pellets have prepared as above. This procedure has been repeated continuously for 10 successive days.

### EXAMPLE 3

### EDTA-Lysozyme in Tris-Phage Breeding Technique

The main objective of the phage breeding techniques pursued was to facilitate phage recognition and clipping onto bacterial cell wall. Crippling of the bacterial cell wall was achieved by using lysozymes.

Standardizing tests were performed in order to establish the optimal breeding formula of lysozyme-EDTA sub-lethal crippling of E. coli cell wall to facilitate the phage clipping and nucleic acid injection into host bacteria. Standardization was categorized into two groups; lysozyme-EDTA action takes place within LB culture directly, and lysozyme-EDTA action takes place with 12 mM Tris-HCl buffer. At both sets of experiments, 1 mM EDTA was used and as follows:

Transfer 100 μl of 10, 15, 50, 100, 500, 1000, 1500, 2000, and 3000 μg ml⁻¹ of lysozyme (Sigma, USA) prepared in distilled water into 1.5 ml sterile microcentrifuge tubes containing: (1) 900 μl of 8 h LB cultures of E. coli clinical isolates and the representative reference E. coli strains, supplemented with 1 mM EDTA or (2) 900 μl of 1 mM EDTA and 12 mM Tris-HCl buffer (pH 8) contain bacterial pellets of 8 h LB cultures, E. coli clinical isolates and E. coli ATCC strains, prepared as above (2. EDTA-Tris buffer-based breeding). Therefore the final lysozyme concentrations in both above mixture are 1, 1.5, 5, 10, 50, 100, 150, 200, and 300 μg ml⁻¹, respectively.

Final concentrations of lysozyme-supplemented EDTA-LB culture were incubated at 37° C. for 18 h were studied. The results from the viable plate count (CFU) revealed that the lysozymic activity of all above mentioned concentrations was insufficient to inhibit the growth of all bacterial strains. In contrast, EDTA at 1 mM, Tris-HCl buffer at 12 mM combined with lysozyme at 200 and 300 mg ml⁻¹ was sufficient to totally inhibit the growth of the tested bacterial strains at pH 8.0. Whilst, only 1-2 logs reduction of CFU observed with all strains in the presence of lysozyme at 150 mg ml⁻¹. Therefore, this concentration was considered as a sub lethal dosage in which the bacterial cells undergo partial destruction of the cell wall to a limit sufficient for surviving. This status is considered ideal for exposing bacteria to a high number of phages that their clipping activity is optimized as bacterial cell wall became brittle.

Therefore, the final formula of the lysozyme-EDTA-Tris phage breeding solution was as follows:

Transfer into 1.5 ml sterile microcentrifuge tubes 600 μl of 20 mM (final concentration 12 mM) Tris-HCl buffer (pH 8), 100 μl of 10 mM EDTA (final concentration 1 mM), 100 μl of 1.5 mg ml⁻¹ of lysozyme (final concentration 150 μg ml⁻¹), 100 μl of 18 hr LB culture of E. coli (1×10⁹ CFU ml⁻¹) and 200 μl of a mixture of different 20 phages (10¹² PFU ml⁻¹) mixed gently and incubated at 37° C. for 10 days with subsequent addition of loopful of 18 h LA culture of E. coli and 100 μl of the desired phages (10¹² PFU ml⁻¹) every 3 days.

The aim of this technique is to find out whether there will be a new bred phage(s) appeared at the end of the 10 rounds of breeding. Mixing of high number of 20 or more different phage strains with high number of crippled bacteria together at favourable long lasting breeding conditions might largely favour the clipping of phages onto E. coli EDTA-caused porous and brittle outer membranes as well as facilitate the nucleic acid injection of phages into the interior of the bacterial host through brittle and highly porous cell wall (due to the effect of EDTA+lysozyme). The advantage of using lysozyme-EDTA over the EDTA alone in the horizontal breeding might be justified that the lysozyme-injured cell wall could allow the loosely attached phages to the outer membrane to inject the nucleic acid successfully in a way difficult to occur when the cell wall was intact.

### EXAMPLE 4

### EDTA-Nisin A in Tris-Phage Breeding Technique

The present inventors tested Nisin A in the phage breeding techniques for E. coli bacteria.

After lengthy pilot studies, the optimal Nisin A (Sigma, USA) concentration was determined after a series of 6 serial dilutions, 0.1 μg ml⁻¹, 1 μg ml⁻¹, 10 μg ml⁻¹, 100 μg ml⁻¹, 200 μg ml⁻¹, and 400 μg ml⁻¹. The breeding mixture used was composed of the above mentioned dilutions of Nisin A at 20 mM Tris, 20 mM EDTA and 1% Tween 20. It was shown that the concentration of a 200 μg ml⁻¹ of Nisin A and above showed a remarkable antibacterial activity against the Gram negative E. coli bacteria. Hence, 100-150 μg ml⁻¹ was decided to be used as the breeding concentration of Nisin A which is able to weaken the E. coli cell wall without a remarkable bacterial destruction. The phage breeding mixture formula was as follows:

Transfer into 1.5 ml sterile microcentrifuge tubes 850 μl of 23.6 mM (final concentration 20 mM) Tris-HCL buffer (pH 8), 20 μl 1000 mM (final concentration 20 mM) EDTA, 10 μl Tween 20 (final concentration 1%), 10 μl of 8 hr LB culture of E. coli (1×10⁹ CFU ml⁻¹), 10 μl of a mixture of high titre 20 desired phages (10¹² PFU ml⁻¹) and 100 μl of 1.5 mg ml⁻¹ (final concentration 150 μg ml⁻¹) of Nisin A. Mixed gently and incubated at 37° C. for 10 days with subsequent addition of loopful 18 h LA culture of E. coli and 10 μl of the desired phages (10¹² PFU ml⁻¹) every 3 days.

### Transmission Electron Microscopy

Transmission electron microscopy (TEM) described by Jassim et al. (2005) was used for some selected phage suspensions with minor modification in brief: 10-20 μl of 2% aqueous phosphotungstic acid (adjusted to pH 7.3 using 1N NaOH) were applied on the phage-adsorbed grids and left on for 3-5 minutes. Then excess fluid was drawn off from the edge of the grid with filter paper. Then electron microscopy was viewed on a Philips CM 200 (Philips Electronics, Holland) at magnifications ranged from 75000× to 160000×.

Since the present invention has designed hundreds of phages and viewing all phages by TEM to get an overall outlook for the characteristics, physical attributes, and the classification of the involved phages are extremely costly and time consuming, representative phage isolates were selected according to two parameters:

1. The host bacterial E. coli strain (Generic or EHEC strains).

2. The geographical area where the phage was isolated.

The phage samples chosen for viewing were arranged in two ways:
a) Pure phage suspensions composed of 2×10⁹ PFU ml⁻¹ of phage particles in lambda buffer solution.
b) Mixed phages-bacteria complexes to disclose the direct contact sites and view the phage interaction directly with the relevant host bacterial cell was carried-out according to Schade et al. (1967) method and in brief as follows: Bacteria were grown to 2×10⁶ CFU ml⁻¹ in LB at 37° C. to produce well-flagellated host cells. A pre-warmed (37° C.) 500 μl sample of 2×10⁹ PFU ml⁻¹ of phage isolate in LB was transferred to 15 ml sterile test tube containing 4.5 ml of 2×10⁶ CFU ml⁻¹ of 6 h LB culture of an appropriates E. coli strains to obtained ratio of 100:1 phage:bacteria. Adsorption was allowed to occur with gentle rotary shaking 30 rev min⁻¹ at 37° C. for 5 minutes. The incubation was terminated by swirling the test tube in ice to chilled bacteria-phage mixture and then the mixtures were filtered through Whatman (Whatman PLC., UK) syringe sterile filter membrane 25 mm/0.22 μm units. The filter washed 3 times with 1 ml of chilled lambda buffer and finally transferred into 15 ml sterile test tube and whereas the trapped bacteria-phage complexes were recovered from filter by gentle hand shaking with 3 ml of the chilled lambda buffer to be ready for negative staining and TEM viewing.

### Results

Isolation and Characterization of E. coli.

Four hundred and thirty, 430, clinical isolates of diagnostically-proven pathogenic E. coli bacteria were retrieved from hospital inpatients (microbiology laboratories, Hospital Serdang and Hospital Kajang in Selangor, Malaysia) from documented sporadic cases of haemorrhagic colitis, non-haemorrhagic colitis, urinary tract infections, infected wounds, vaginitis and bacteremic cases. Several morphologically distinct types of colonies were apparent on the LA plates used for determining the bacterial cell count. Representative samples of each were transferred with sterile toothpicks into liquid LB broth. The isolates were re-checked and identified by using Microbact GNB 12A system (Oxoid, UK) with 99% confirmatory diagnosis for E. coli. In addition, EHEC isolates were identified by using sorbitol MacConckey agar test. It was found that 413 (96.05%) of the involved clinical isolates fermented sorbitol, namely, they are non-EHEC, save for 17 clinical isolates (3.95%) (Table 1) were sorbitol non-fermenter, therefore, they were considered as EHEC

All E. coli clinical isolates and reference E. coli NTCC 129001, NTCC 9001, ATCC 12810, ATCC 12799, ATCC 25922, and ATCC 35218 strains were subjected to be the host targets for the isolation of wild phages, phage redesign and breeding (Table 1).

### Phage Isolation, Optimization, and Redesign Techniques

One hundred and forty nine (149) highly lytic and specific E. coli phages isolates have been retrieved from wild and redesigned via vertically breeding (gain optimization), and/or horizontally bred (earn new specificity). 121 phages have been vertically bred (Table 1) whereas 19 phages were developed from 6 reference strains (NTCC 129001, NTCC 9001, ATCC 12810, ATCC 12799, ATCC 25922, and ATCC 35218), 92 phages were developed with 143 non-EHEC clinical isolates, 10 phages were obtained from 10 EHEC clinical isolates cultures and 13 phages for EHEC represent 10 phages from clinical isolates and 3 phages developed on one single EHEC NTCC 129001.

However, some phages were found completely resistant to culture on various E. coli strains have been developed further to gain prototype highly specificity via horizontal breeding techniques (Table 1) whereas 22 phages were obtained from 22 E. coli strains (16 non-EHEC and 6 EHEC) and 6 phages were bred on 5 reference strains non-EHEC and 1 EHEC. In general, the total phages have been successfully horizontally bred and yielded with highly prototype specificity were 28 phages in which 7 phages are EHEC-specific phages and they did not respond to the vertical breeding techniques.

Accordingly, a huge coliphage mixture was built gradually and called phage master mix. Upon the build up of phage master mix, an increasing number of the bacterial isolates were immediately recognized and lysed by this mixture without the need to isolate or breed new phages therefore the number of the isolated/bred phages, 149, is smaller than the total number of host cells, namely the clinical isolates and the reference strains. When the phage master mix was finally composed of 149 phage isolates, it covered >95% of any given number of pathogenic E. coli isolates see Table 1, which shows the demographic estimates of the E. coli clinical isolates, reference E. coli strains, crude samples for phage isolation, and the bred phages developed.

The retrieved phage isolates showed a remarkable variation in the plaques morphology, plaques size, plaques clarity, phage titre, and other phage biokinetic tributes. However, since there is a possibility of E. coli strain overlapping among the studied bacterial isolates.

TABLE 1

| | |
|---|---|
| The clinical | Total E. coli number: 430 |
| isolates | No. of non-EHEC isolates: 413 |
| | No. of EHEC isolates: 17 |
| | % of EHEC isolates: 3.95% |
| | Source of isolates: 70% stool of patients, 30% (urine, |
| | blood and vagina) of human patients. |
| | No. of isolates yielded new phages by vertical |
| | breeding: 153 |
| | 143 non-EHEC |
| | 10 EHEC |
| | No. of isolates yielded new phages by horizontal |
| | breeding: 22 out of 24 clinical isolates underwent |
| | horizontal breeding: |
| | 16 out of 17 non-EHEC |
| | 6 out of 7 EHEC |
| | Total No. of clinical isolates yielded new phages |
| | by both vertical and horizontal breeding: 153 + |
| | 22, respectively, = 175 |
| | The rest of isolates 241 were readily covered by phages |
| | produced and bred from the above 175 isolates |
| | Total no. of covered isolates by bred phages: 175 + |
| | 241 = 416 |
| | The final resistant isolates: 430 − 416 = 14 |
| | isolates only |
| | % of covered E. coli isolates by bred phages: 96.7% |
| The reference | Total no.: 6 reference strains. |
| strains | Non-EHEC: 5 strains (NTCC 9001, ATCC 12799, ATCC |
| | 12810, ATCC 25922, and ATCC 35810) |
| | NTCC 9001: yielded 7 vertically bred phages and 1 |
| | horizontally bred phage. |
| | ATCC 12810: yielded 3 vertically bred phages and 1 |
| | horizontally bred phage. |
| | ATCC 12799: yielded 2 vertically bred phages and 1 |
| | horizontally bred phage. |
| | ATCC 25922: yielded 2 vertically bred phages and 1 |
| | horizontally bred phage. |
| | ATCC 35810: yielded 2 vertically bred phages and 1 |
| | horizontally bred phage. |
| | EHEC strains: 1 strain (NTCC 129001) |
| | NTCC 129001: yielded 3 vertically bred phages and 1 |
| | horizontally bred phage. |
| The crude | Sources: animal stool (sheep, cow, horses, camel, quell, |
| samples | chicken, birds), manure, soil, and sewage. |
| for phage | No. 113 different crude samples |
| isolation | Each 8 samples mixed together to form crude mixtures |
| | Each run: mixing of a crude mixture, composed of 8 |
| | crude samples + 10 (or more) clinical E. coli isolates |
| The bred | No. of phages: 140 E. coli specific phages developed |
| phages | from the clinical E. coli isolates and from the |
| | reference strains |
| | 121 phages were isolated and vertically bred. |
| | 19 phages isolated/vertically bred from 6 reference |
| | strains, 3 of which isolated from EHEC reference strain. |
| | 92 phages isolated/vertically bred from 82 non-EHEC |
| | clinical isolates. |
| | 10 phages isolated/vertically bred from 10 EHEC clinical |
| | isolates. |
| | 28 phages were horizontally bred successfully from 30 |
| | E. coli bacteria |
| | 22 phages were bred from 16 non-EHEC and 6 EHEC |
| | isolates. |
| | 6 phages were bred from reference strains (5 non-EHEC |
| | and 1 EHEC). |
| | 7 phages out of the above-mentioned 28 horizontally |
| | bred phages were EHEC-specific phages. |
| | The success rate of the horizontal breeding: |
| | 28/30 = 93.3% |
| | The total No. of the EHEC-specific phages isolated, |
| | vertically bred, and horizontally bred: 13 + |
| | 7 = 20 phages |

### Vertical Breeding

The phages that have been isolated and bred from the reference generic E. coli NTCC 9001, ATCC 12810, ATCC 12799, ATCC 25922, and ATCC 35218 strains were numbered and abbreviated as (G) and the phages isolated from the reference EHEC E. coli NTC129001 strain were numbered and abbreviated as (H), while the phages isolated and bred from the clinical isolates were numbered according to the relevant clinical isolate. See Table 2A which shows the vertical breeding and optimization of different phages isolated from wild and bred on five non-EHEC reference strains and one EHEC reference strain showing optimized plaque and biokinetic values, and Table 2B which shows the difference between the observation frequency of clear (CL) and semi-clear (SC), semi-turbid (ST), and turbid (TR) plaques before and after vertical breeding.

The results of the vertical breeding and optimization for the isolated phages were highly promising in terms of the phage plaque criteria and in the phage biokinetic values. Since the biokinetic values, the burst time (BT) and the optimal phage binding time (PBT) showed no remarkable differences before and after breeding, only the burst size (BS) and the infective ratio (IR) were shown in Tables 2 and 3.

Regarding the phages isolated and vertically bred from the reference NTCC and ATCC strains of E. coli, it was found that the mean of the phage plaque size before breeding, 1.87 mm, is much lower than that of the optimized phages, 4.26 mm (P<0.01), Table 2A. The observed clarity of the plaques in the post-breeding phages was associated more with clear (CL) plaques than the pre-breeding phages (P<0.01), Table 2B. The mean of the burst size (BS) in the pre-breeding phages, 174.1, was lower than that of the post-breeding phages, 288.47 (P<0.01), Table 2A. And the mean of the infective ratio (IR) of the pre-breeding phages, 79.93, was lower than that of the post-breeding phages, 91.32 (P<0.01), Table 2A. The results obtained from the vertical breeding on the clinical E. coli isolates was similar to that obtained from the vertical breeding of the reference E. coli strains. See Table 3 which shows vertical breeding and optimization of different phages bred on 153 E. coli clinical isolates that composed of 143 non-EHEC and 10 EHEC. Moreover, the increments in the IR, BS, and plaque size values after the breeding were correlated positively with each other. It was found that the correlation coefficient between the increments of IR and BS was r=+0.4, and between the increments of BS and plaque size was r=+0.35, and between the increments of IR and plaque size was r=+0.3 (P<0.05). This provided further consistency of our optimization techniques that three parameters for the phages lytic cycle optimized similarly and correlated with each other significantly. The harmony in the optimization of these parameters, namely, the infective ratio, the burst size, and the plaque size represents that the optimized phages have been enhanced in respect to their host infectivity, their replicative potential inside the host, and their lytic activity as well.

TABLE 2A

| | | | | Before breeding | | | | After breeding | | | |
|---|---|---|---|---|---|---|---|---|---|---|---|
| Reference | Type the of | | | Plaques | | Biokinetic | | Plaques | | Biokinetic | |
| strain of | reference | Crude specimen | Phage | Size | | IR | | Size | | IR | |
| E. coli | strain | of the phage | name | (diameter; mm) | Clarity | (%) | BS | (diameter; mm) | Clarity | (%) | BS |
| NTCC 9001 | Non-EHEC | Camel stool | 2G | 2 | SC | 94 | 81 | 5.5 | CL | 97 | 255 |
| | | Pigeon litter | 4G | 3 | CL | 89.3 | 232 | 6.5 | CL | 94.3 | 316 |
| | | Chicken litter | 5G | 4 | SC | 88.6 | 130 | 7 | CL | 95.1 | 278 |
| | | Sheep stool | 6G | 3 | CL | 90 | 108 | 6 | CL | 98 | 315 |
| | | Manure | 8G | 5 | ST | 75 | 73 | 7 | CL | 89.8 | 219 |
| | | Chicken litter | 9G | 2.5 | SC | 43 | 130 | 6 | CL | 85 | 287 |
| | | Chicken litter | 10G | 2 | SC | 82.2 | 148 | 4 | CL | 94 | 294 |
| ATCC 12810 | Non-EHEC | Sheep stool | 11G | Invisible 0.1 | TR | 68.4 | 187 | 2 | SC | 84 | 305 |
| | | Cow stool | 12G | 1 | ST | 72.7 | 213 | 2.5 | SC | 82.5 | 288 |
| | | Goat stool | 13G | 1.5 | SC | 86.1 | 246 | 3 | CL | 90.2 | 324 |
| ATCC 12799 | Non-EHEC | Farm soil | 15G | Tiny 0.3 | ST | 72.8 | 153 | 2 | CL | 88.6 | 276 |
| | | Chicken litter | 16G | 1.5 | SC | 83.5 | 231 | 4 | SC | 94.2 | 284 |
| ATCC 25922 | Non-EHEC | Quell litter | 20G | 2.5 | CL | 91.7 | 286 | 5 | CL | 95.8 | 304 |
| | | Chicken litter | 21G | 2.5 | SC | 88.3 | 245 | 5 | CL | 93.9 | 295 |
| ATCC 35218 | Non-EHEC | Sheep stool | 24G | 2.5 | CL | 91.2 | 268 | 4 | CL | 95 | 326 |
| | | Manure | 25G | 2 | SC | 85.6 | 274 | 4 | CL | 93.1 | 286 |
| NTCC 129001 | EHEC | Pigeon litter | 4H | Invisible 0.1 | TR | 78.8 | 101 | 2 | CL | 87.3 | 273 |
| | | Chicken litter | 9H | Invisible 0.01 | TR | 65.8 | 110 | 3 | CL | 86.9 | 289 |
| | | Sheep stool | 10H | Invisible 0.01 | TR | 71.7 | 92 | 2.5 | CL | 90.4 | 267 |

-CL: clear plaque -SC: semi-clear plaque -ST: semi-turbid plaque -TR: Turbid plaque -BS: Burst size -IR: infective ratio

TABLE 2B

| Variables | CL | SC, ST, TR | Total |
|---|---|---|---|
| pre-breeding | 4 | 15 | 19 |
| Post-breeding | 16 | 3 | 19 |
| Total | 20 | 18 | 38 |

-CL: clear plaque
-SC: semi-clear plaque
-ST: semi-turbid plaque
-TR: Turbid plaque
-BS: Burst size
-IR: infective ratio

TABLE 3

Phages isolated and vertically bred from
non-EHEC clinical isolates only:
No. of phages: 92
No. of clinical isolates: 143
No. of phages showed plaque size increase
after the vertical breeding:
Plaque size (diameter) increase >5 mm: 14 phages
Plaque size (diameter) increase 4-5 mm: 40 phages
Plaque size (diameter) increase 2-3 mm: 32 phages
Plaque size (diameter) increase 0.5-1 mm: 6 phages

| Studied parameter | Before breeding | After breeding |
|---|---|---|
| Average of the plaque size | 2.3 mm | 4.12 mm |
| Significant increase (P < 0.01) | | |
| Plaques clarity | 37 CL | 67 CL |
| Significant increase (P < 0.01) | 23 SC | 10 SC |
| | 12 ST | 4 ST |
| | 11 TR | 2 TR |
| Average of the biokinetic value (IR) | 81.3 | 92.6 |
| Significant increase (P < 0.01) | | |
| Average of the biokinetic value (BS) | 204.7 | 316.0 |
| Significant increase (P < 0.01) | | |

Phages isolated and vertically bred from
EHEC E. coli clinical isolates:
No. of phages: 10
No. of clinical isolates: 10
No. of phages showed plaque size
(diameter) increase:
>5 mm: 0
4-5 mm: 4
2-3 mm: 5
0.5-1 mm: 1

| Parameter studied | Before breeding | After breeding |
|---|---|---|
| Average of the plaque size | 1.8 mm | 3.7 mm |
| Significant increase (P < 0.01) | | |
| Plaques clarity | 2 CL | 6 CL |
| Significant increase (P < 0.01) | 3 SC | 2 SC |
| | 2 ST | 2 ST |
| | 3 TR | 0 TR |
| Average of the biokinetic value (IR) | 77.4 | 89.2 |
| Significant increase (P < 0.01) | | |
| Average of the biokinetic value (BS) | 211.8 | 286.5 |
| Significant increase (P < 0.01) | | |

-CL: clear plaque
-SC: semi-clear plaque
-ST: semi-turbid plaque
-TR: Turbid plaque
-BS: Burst size
-IR: infective ratio

### Phage Biokinetics

Phage growth was characterized by the latency period, the burst size and by the percentage of adsorption to the host cells after 1, 5, 10 and 15 min (determined all in modified one-tube growth experiments).

The results showed that all isolated phages from the vertical breeding (Tables 1 and 2) have an optimal phage binding to host cell of 5 to 10 min with the burst time of 25 to 40 min with non-significant difference between the phages before and after breeding (P>0.05). On the other hand, the burst size showed great variance among the tested bred phages and showed a significant difference between pre- and post-breeding phages, as mentioned earlier. The minimal burst size was 73 phage particles per a cycle and the maximal burst size was 336 phage particles per cycle. One of the most important parameters of the phage biokinetics is the infective ratio (IR) in which it was found highly variable among the tested phages as well as significantly different between pre- and post-breeding phages, as mentioned earlier. Nevertheless, all E. coli strains have shown partial or complete phage lysis resistance have been subjected to a series of phage horizontal breeding process.

### Horizontal Breeding

Three horizontal phage breeding techniques were applied on 6 E. coli reference strains and on 24 clinical isolates that showed great, unbeatable resistance against all isolated and optimized phages obtained in this study to determine whether these techniques can result in phages conferring new host range specificity. The vital factors that lead to the success of the current horizontal breeding techniques were; (1) using a large number of different wild isolated phages per each run of breeding, (2) using phages were previously vertically bred and highly optimized on other E. coli strains, (3) preparing suitable microenvironment conditions for the horizontal breeding techniques to bias the co-evolutionary balance between phages and bacteria towards the phages. In general, it was found that the results from using only a single or a couple of phages against highly phage-negative cultures of E. coli were disappointing. Therefore, it was believed that using larger numbers of isolated/optimized phages for single target resistant bacteria would give much better results. Accordingly, 20 highly optimized E. coli-specific phages, each being 100% non-specific for the 30 bacterial strains/isolates used, were involved in the three techniques of the horizontal breeding of Examples 2, 3 and 4. These phages were selected as; a) highly optimized coliphages with large clear plaques and high IR %, b) non-specific to any of the reference strains used in the experiments and non-specific to any of the 24 highly phage-resistant clinical isolates, 3) different phages in terms of plaque morphology and biokinetic criteria. Therefore, 30 reaction tubes of horizontal breeding, each tube contains one target bacteria with 20 phages, were used for each technique of the breeding. The 90 reaction tubes for the 3 used techniques were accompanied with 30 negative control tubes which each contains a mixture of one target bacterium with the same 20 phages in Tris-buffer without adding the horizontal breeding reagents, EDTA, lysozyme, Nisin A, or Tween 20.

Twenty eight new specific phages were obtained towards 28 previously phage-resistant bacteria by using simultaneously 3 horizontal breeding techniques (Tables 4 and 5). All reference strains, five non-EHEC and one EHEC, and 22 clinical isolates, 16 non-EHEC and 6 EHEC bacteria, resulted in one new phage for each, totally 28 phages. Twenty one new phages were produced successfully by only one breeding technique while the rest of phages were produced by two breeding techniques. Nevertheless due to the great similarity between the two phages coming out from the breeding techniques, they were considered as one phage. The results of the horizontal breeding (Table 4 and 5) showed that it is possible to confer new specificity for non-specific phages toward certain target bacteria when favouring breeding conditions are sustained for a long period of time and for many successive runs. The newly bred phages produced initially 1 mm diameter semi-turbid plaques on bacterial lawns, however, with subsequent series of frequent vertical breeding, the plaques diameter have enlarged to 2-3 mm in diameter and furthermore became highly transparent. It was inferred that if one of the breeding techniques had successfully developed a bred phage for a host cell, it doesn't mean that the same protocol will work with other strains and this has inspired to use all three phage horizontal breeding techniques simultaneously for all highly phage resistant E. coli strains (Tables 4 and 5). None of the negative control reactions (absence of EDTA, lysozyme, Nisin A, or Tween 20) showed any new phages against any of the 30 resistant E. coli strains used. This granted a solid base on the possible mechanisms responsible for the horizontal breeding that required necessarily the presence of chelating, detergents, and cell wall destroying agents like EDTA, lysozyme, Nisin A, and Tween 20.

TABLE 4
Horizontal phage breeding on 30 E. coli strains:
6 reference strains and 24 clinical isolates.

| | | Chemical treatment | | |
|---|---|---|---|---|
| E. coli | Type of | | EDTA- | EDTA- |
| strain | E. coli | EDTA | lysozyme | Nisin |
| NTCC 9001 | Non-EHEC | − | + | + |
| ATCC 12810 | Non-EHEC | − | + | + |
| ATCC 12799 | Non-EHEC | + | − | − |
| ATCC 25922 | Non-EHEC | − | + | − |
| ATCC 35810 | Non-EHEC | − | − | + |
| NTCC 129001 | EHEC | + | + | − |
| 1 | Non-EHEC | + | − | − |
| 2 | Non-EHEC | − | + | − |
| 5 | Non-EHEC | − | − | + |
| 14 | EHEC | − | + | − |
| 31 | Non-EHEC | + | − | − |
| 84 | Non-EHEC | − | + | − |
| 91 | EHEC | − | + | + |
| 78 | Non-EHEC | − | − | − |
| 8 | Non-EHEC | + | + | − |
| 12 | Non-EHEC | − | − | + |
| 22 | EHEC | − | + | − |
| 24 | Non-EHEC | − | − | + |
| 34 | EHEC | − | − | + |
| 48 | Non-EHEC | + | + | − |
| 128 | EHEC | − | − | − |
| 75 | Non-EHEC | − | − | + |
| 111 | Non-EHEC | − | + | − |
| 113 | Non-EHEC | − | − | + |
| 127 | Non-EHEC | − | − | + |
| 133 | EHEC | − | + | − |
| 159 | EHEC | + | − | − |
| 160 | Non-EHEC | − | + | + |
| 180 | Non-EHEC | − | + | − |
| 191 | Non-EHEC | − | − | + |

| | | | |
|---|---|---|---|
| Total bred phages | 7 | 15 | 13 |

TABLE 5
A summary results of the phage horizontal breeding techniques.

| No. of phage breeding | Three protocols: | |
|---|---|---|
| protocols used for each E. coli | Tris-EDTA phage | (a |
| strains | breeding technique. | |
| | Tris-EDTA-lysozyme | (b |
| | phage breeding | |
| | technique. | |
| | Tris-EDTA-Nisin A- | (c |
| | Tween 20 phage | |
| | breeding technique | |

| | |
|---|---|
| Total no. of highly resistant, | 30 E. coli strains |
| namely, phage negative | 5 reference non-EHEC strains. |
| culture E. coli used | 1 reference EHEC strain. |
| | 17 non-EHEC clinical isolates. |
| | 7 EHEC clinical isolates |
| No. of the responsive E. coli | 28/30 |
| isolates to horizontal bred | |
| phage | |
| Success rate of phage | 93.3% |
| breeding (%) | |
| No. of the responsive E. coli | 7/30, (23.33%) |
| isolates and the success rate | |
| for the phage breeding Tris- | |
| EDTA | |
| No. of the responsive E. coli | 15/30, (50%) |
| isolates and the success rate | |
| for the phage breeding | |
| technique Tris-EDTA- | |
| lysozyme | |
| No. of the responsive E. coli | 13/30, (43.33%) |
| isolates and the success rate | |
| for the phage breeding | |
| technique Tris-EDTA-Nisin | |
| A-Tween | |
| The Number of phages used | 20 highly optimized lytic phages were |
| in each of the 3 techniques | used in mixture and co-cultured with |
| of breeding | the target resistant isolate |
| Number of successive days | 10 successive days |
| (rounds) of breeding | |
| No. and % of the responsive | 21/22 E. coli strains 95.45% (5 reference |
| non-EHEC E. coli to breeding | strains and 16 clinical isolates) |
| No. and % of the responsive | 7/8 E. coli strains 87.5% (1 reference |
| EHEC E. coli to breeding | strain and 6 clinical isolates) |
| No. and % of the resistant | 2 E. coli strains 6.6% (one EHEC and one |
| E. coli bacteria to | non-EHEC clinical isolates) |
| horizontal breeding | |

### TEM

From the TEM micrographs, the selected phages showed a great diversity in respect to their physical characteristics (Table 6) and they were classified into different T-series according to Brock (1990). The phage master mix shows a great hybrid of optimized/bred/isolated anti-E. coli phages that belong to the all known phage T-series. This ensures the high diversity of the phage mixture which in turn ensures the highest possible E. coli coverage and effectiveness. Eight phages out of ten tested showed tendency to attach to somatic O antigens rather than to flagellar H antigens. Therefore, it is unlikely that the expression of H7 antigen plays a role in plaque resistance since several other O157 non-H7 strains were susceptible to plaque formation by the phages (Kudva et al., 1999; Goodridge et al., 1999 and 2003). It can be inferred that LPS (O antigen) is the most crucial element determining the phage-host specificity, so effective phage infection into resistant host might require modified LPS, namely O antigen. This is supported by Mizoguchi et al., (2003) who revealed that phage mutants seemed to originate by alternation of LPS structure.

TABLE 6
Classification and characterization of selected
designed phages from electron micrographs

| Phage | | | Size (nm) | |
|---|---|---|---|---|
| bred | | | Head | |
| number | E. coli type | Phage series | (Diameter) | Tail |
| 4G | Non-EHEC | T5 | 95; circular | 22 |
| 4H | EHEC | T5 | 102; circular | 45 |
| 8G | Non-EHEC | T3 or T7 | 35; circular | 45 |
| 9H | EHEC | T5 | 105; circular | 63 |
| 10H | EHEC | T-even 2, 4, 6 | 88; icosohedral | 105 |
| 175 | Non-EHEC | T-even 2, 4, 6 | 90; circular | 85 |
| 115 | Non-EHEC | T3 or T7 | 53; circular | 80 |
| 131 | EHEC | T1 | 65; icosohedral | 85 |
| 91 | EHEC | T-even 2, 4, 6 | 30; circular | 80 |
| 15G | Non-EHEC | T5 | 80: oval | 55 |

### Discussion

E. coli Clinical Isolates

Seventy percent of E. coli clinical isolates were obtained from patient stool samples with gastrointestinal tract disorders like diarrhoea, abdominal pain, food poisoning, and enterocolitis whilst, the other 30% were found in patients' urine, blood and vaginal swab samples. Not surprisingly by E. coli infection for human beings is usually transferred from the environment and more importantly from surrounding animals. E. coli are usually present in the bowel of the warm-blooded animals and particularly in the livestock of cattle, sheep, horses, camels, chicken, cats, dogs and birds (Jackson et al., 1998; Garber et al., 1999; Milne et al., 1999). Disease-causing microbes that have become resistant to drug therapy are an increasing public health problem and E. coli O157:H7 and MRSA are examples of the diseases that have become hard to treat with antibiotic drugs.

### Unprecedented Achievements

The described protocols to produce highly reliable phage or phage cocktail with high specificity able to infect and lyse wide ranges of E. coli that cause gastroenteritis in humans including EHEC strains. One of the important steps of the current invention is to calculate the lytic cycle kinetics of the isolated and bred phages. This step is mandatory for the subsequent applications based on the discovered phages, including; phage-based rapid diagnostics, phage-based biocontrol and bioprocessing or in phage therapy for E. coli infections. In this invention the present inventors have formulated a new phage biokinetics measurement by using only one single tube of assay.

In the present invention of non-genetic phage designing techniques which succeeded in breeding wild phages to acquire optimized infective traits, “vertical breeding”, and to acquire new traits that had never been reported previously, “horizontal breeding”. Therefore, the present invention presents first evidence to formulate a phage master mix isolated from the wild environment and bred/redesigned by the described techniques to cover >95% of all pathogenic E. coli strains.

### Novel Phage Vertical Breeding and Phage Biokinetics

It was postulated that successful phage-based applications for example therapy, bacterial detection, biocontrol and bioprocessing could be achieved by, firstly finding a reliable method of hunting a large number of wild phages in a short time, secondly establishing a method to enhance and promote the lytic characteristics of the isolated phages, and thirdly finding a method to exploit the large number of the optimized isolated phages to infect unrecognized strains by designing new prototype phages with new host specificities. Therefore, two kinds of breeding technologies were designed as described above producing highly optimized E. coli-specific prototype phages which cover almost all of the important strains of E. coli with more than 3-5 optimized specific phages for each strain.

Analyzing deliberatively the phases of the lytic cycle of each phage is crucial and vital for any phage-based bacterial diagnostic, therapy, biocontrol and bioprocess protocols. Without knowing the biokinetic criteria of phages precisely, it would be impossible to manipulate phages toward the desired host target. The fact that a single tube could harbour all the encountering bacteria and phages together without the need to use another tube is a guarantee for the accuracy and preciseness. Furthermore, spotting onto a bacterial lawn of the host bacteria is simpler than using plaque semi-solid top layer agar assay. Using the biokinetic tests, a solid base was issued in designing accurately the future phage-based bacterial diagnostic tools which should be congruous with the lytic cycle of the phages implemented.

Hence, the protocol described herein for measuring precisely the phage biokinetics in simple single test tube could act as the template procedure for all redesigned phages.

The results of the different phases of the vertical breeding were very encouraging. It has been shown that the increments of plaques size after the breeding, along with clearer plaques, was about 2.5 mm increase for the reference strains, 1.82 mm increase for the non-EHEC clinical isolates, and 1.9 mm increase for the EHEC clinical isolates (P<0.01). The post-vertical breeding increase of IR, about 11%-12%, was found in the reference strains, non-EHEC isolates, and EHEC isolates (P<0.01). The post-vertical breeding increase of BS in both reference strains and the non-EHEC isolates was about 112-114 (P<0.01), while the post-vertical breeding increase of BS in EHEC isolates was lower, about 75, but still significant increase (P<0.01). These results have proved definitely that the plaque-based and biokinetic-based approaches of phage vertical breeding are highly successful in deploying effective phage against E. coli bacteria or against any other target bacteria. The optimized phages showed remarkably higher potentials of bacterial infectivity, better host specificity, more aggressive lytic kinetics, and higher replicative standards.

It was shown that all tested phages after vertical breeding were of good burst size 73-336 with an optimal phage binding time of less than 10 minutes. The burst time was universally around 25-40 minutes and the range of IR was 74% to 98%. The biokinetic values of the burst time (BT) and the optimal phage binding time (PBT) showed no remarkable differences before and after breeding, only the burst size (BS) and the infective ratio (IR) did. This might be difficult to be explained but it was conceived that BT and PBT might be associated more with the T-family phage classification rather than to the phage optimization. Accordingly, IR, which reflects principally the specificity and the affinity of the attacking phages to their host cells, has reflected a good parameter for the post-vertical breeding optimization level. Alike, BS showed a similar good response to the optimizing techniques pursued. This might be attributed to the optimization of the recognition/specificity of the attacking phages to their host cells which leads to more stably bind phages to the host in a way that multiple phages can get inside a single host cell and amplify more effectively, or attributed to the activation of some early enzymes (EA) of the attacking phages which lead to higher replicative phage cycle.

Whilst, vertical breeding relied mainly on the accumulative bias in the selection of the minutely larger plaques of hunting the clones of phages underwent some kind of beneficial somatic changes. These somatic changes are though to be driven by certain mutations which are probably single base mutations in the genes encoding for tail fibre recognition sites, genes of lysozyme excretion, or genes of early phase enzymes which deploy the host metabolism for the phage tactics.

A significant positive correlation coefficient was found among the post-breeding increment values of high infective ratios (IR), high post-breeding burst size (BS), and plaque size of the tested phages which gave a clue on the comprehensive nature of the invented optimization techniques. This serves well for formulating a huge mixture of potentially optimized phages against many of E. coli strains or any other bacteria, for preparing the basis of successful horizontal breeding which requires high number of optimized starter phages to give new specificities, and for establishing a background of successful phage rapid diagnostic and phage therapeutic trials.

The IR, BS, the relatively short burst time (BT), and the highly optimized lytic characteristics (larger and much clearer plaques) are the most important parameters for selecting the best phages for designing the diagnostic, therapeutic, biocontrol and bioprocess protocols. Most of designed phages were capable of amplification by 3 logs every 25-40 min, with an average of 30 minutes. Thus it will be the pillar trait of getting high yield phage progenies in which fast and precise diagnostic tests could be attainable using many detection techniques like ATP release, fluorescent dyes, immunological assays etc.

### Horizontal Breeding

The modifications and optimizations resulted from both the vertical and the horizontal breeding are necessary to make up a master phage cocktail that will serve as a template for any given bacteria and at any given geographical region. Upon request, the master phage mixture can be adjusted further to convert phage-negative host cell to positive for lytic phage via horizontal breeding. It's well known that not all bacterial strains are straightforwardly subject to lysis by lytic phages (Kudva et al., 1999). However, the master phage mixture of 20 vertically bred phages were undergone horizontal breeding using three simultaneous techniques; Tris-EDTA, Tris-EDTA-lysozyme and Tris-EDTA-Nisin-Tween. The 20 master phage mixture showed a total success rate of 93.3% (Table 4) by using the 3 techniques simultaneously (50%, 43.3%, and 23.3% for Tris-EDTA-lysozyme, Tris-EDTA-Nisin-Tween, and Tris-EDTA, respectively). Nevertheless, it was found that target bacterial strains and isolates respond differently to each technique which gives a clue that each technique exerts different mechanism of breeding.

The exact mechanism for acquiring new host specificities is still unknown. It is thought that, exposing hidden phage-specific receptors on the host cell, modifying the 3-dimensional configuration of these receptors, or facilitating the entry of the nucleic acid of the phages through brittle cell wall, all lead to the artificially driven intracellular replication of the phages. It is highly probable that a genetic interaction takes place between the naturally non-specific phages and some genetic elements inside the host cell.

EDTA alone, or supplemented with lysozyme or Nisin A, acts as a chelating agent on the bacterial cell wall which can lead to higher membrane permeability, more brittle cell wall or even tiny holes/tears in the outer membrane and cell wall of the target E. coli. This enables the non-specific phages to cross the cell wall and contact the partially-torn peptidoglycan layer.

Bacteriophages usually need 3 tail fibres and more to clip to certain receptors on the cell wall of bacteria in order to start end plate attachment in a stable way and then start phage DNA injection into the host bacteria (Weber et al., 2000). Hence, the outer membrane of EDTA-treated bacteria might become highly permeable and perceptible for phage tail fibres that responsible for the recognition of the host bacteria. Moreover, the configuration of LPS and teichoic acids might be changed, some of the hidden moieties might be exposed which all might have facilitated the clipping of phages into EDTA-treated bacteria leading to abnormally occurring lytic cycle.

Nevertheless, the exact phage infection mechanism is still unknown (Letellier, et al. 2004), but it is believed that LPS-degrading phage enzymes facilitate the penetration of phages and such enzymes have been found as structural elements in Gram negative bacteria phages (Baxa et al., 1996; Steinbacher et al., 1997). Thus the key for successful horizontal phage breeding is modifying the bacterial cell wall using for example chemical treatment of the Examples providing phage access to the interior of the host. Inside the host cell, new information can be obtained from the remnants of current or previous phages (mainly lysogenic) that have infected the target strain of bacteria. In other words, the new phage can obtain new specificity information from other phage genes residing in the chromosomal or plasmid genomic material of the host bacteria. Most Enterobacteracea, including E. coli, are susceptible to hundreds of lytic or lysogenic phages. Therefore, it is rare to find an isolate of E. coli which has not undergone lysogenic phage infections leaving resident prophage(s) dormant inside the cell. These prophages behave as excellent genetic transfer molecules and can change the phenotypic traits of the host cells. The source of these phenotypic changes can be through prophage-encoded toxins, bacterial cell surface alterations, or resistance to the human immune system. Further, prophage integration into the host genome can inactivate or alter the expression of host genes. These resident lysogenic phages are specific phages able to infect this particular strain, but they are unable to conduct a lytic infection due to the lack of lytic cycle genes or what is recently called the “bacteriophage resistome” (Hoskisson and Smith, 2007) including crispr-associated (Cas)-clustered regularly interspaced short palindromic repeats (CRISPR), which comprises clusters of repetitive DNA (CRISPR) that is associated with up to six core cas genes (Edward and Ivana, 2007) whereas, cas-CRISPR implicates in providing a mechanism for integration of bacteriophage DNA fragments into chromosomal sites to promote resistance to future infection: a form of acquired immunity (Barrangou et al., 2007). These defence mechanisms have a profound effect on host range and therefore on the use of phage as biocontrol, bioprocess and therapeutic agents. Hence, this phage design protocol might overcome the defence mechanisms by designing highly specific lytic phages for a particular resistant bacterial strain by using the combined vertical and horizontal breeding to gain the recognition genes which reside inside the host without losing lytic genes of the bred phages. Consequently, it is proposed that phages that were forced or facilitated to insert inside bacterial cells will acquire new specificity genes from the non-lytic resident temperate phages present inside the bacterial host, and at the same time not lose their lytic genes.

However, it was thought that not all phages in the breeding solution could recognize successfully the newly modified host cells. Moreover after succeeding to get inside the host cell, not all of them could do successful intracellular interaction which is necessary to gain the particular specificity to that host cell. Therefore for highly particular resistant strains “highly phage-negative cultures E. coli”, it was found that higher phage number in the phage master mix leads to higher success rate of the phage infection.

Repetitive cycles of horizontal breeding techniques lead to a phage population with an entirely altered host affinity. The post-breeding phage progenies do not show a distribution of attachment and virulence equivalent to the original population but instead the entire population developed new potential of recognition, attachment and infectivity against the target host cells. It is noted that the post-breeding phage progenies have not been considered successful new phages until they succeeded 100% infective activity on the target negative host culture. This ensures that the post-breeding phage progenies have gained new genetically transferred traits that make them able to recognize and lyse physiologically normal target host cells.

The phage master mix can be produced in any geographical region and is aimed at being sufficient to cover almost all pathogenic E. coli in that region. For example the phages isolated and designed on bacterial isolates from Asia are almost of the same importance as bacteria present in Africa or Europe. Nevertheless, it is postulated that the E. coli phage master mix will be the background of any further refinement suitable for any country, continent or geographical region.

One of the important points of phage breeding programmes is to avoid the development of bacterial resistance towards infective lytic phages. This resistance is considered as the most significant adverse effect of using phages in biocontrol/therapy and in phage-based diagnostics (Merril et al., 1996). One current application of the phage hunting and phage breeding techniques is the production of a reliable phage cocktail able to cover almost all pathogenic E. coli strains, each bacterial strain being recognized by more than one specific designed lytic phage. In this way, if one strain developed resistance to one specific phage in the cocktail, the other phage will compensate the deficit and subdue the resistance development at its very initial stage. This is the same principle as multi-drug therapy towards serious infectious agents such as in bacterial septicaemia.

### Phage Therapy for the Multiple Drug Resistant Bacteria (MDRB)

Phage therapy is simply another form of biological control—the use of one organism to suppress another; and like other biological controls, the application of phage therapy holds a potential to reduce the usage of anti-pest chemicals, which in the case of phages means a reduction in the application of chemical antibiotics. One of the most hindering setbacks of using phages in bacterial therapy has been the development of resistance as described above and the difficulty of finding the suitable alternative phages timely. However, there is now an upsurge of using phages again for therapy (Sulakvelidze et al., 2001), which is on the contrary of antibiotics its arsenal is imperishable, because of the appearance of life-threatening bacterial infections by MDRB like Methicillin-resistant Staphylococcus aureus (MRSA) and Mycobacterium tuberculosis. Therefore, the exploitation of bacteriophages as a realistic approach to the control of pathogens has attracted considerable interest in recent years (Sulakvelidze et al., 2001). Therefore, the key solution to succeed in all mentioned above phage-based applications is to formulate a cocktail of highly specific phages that are able to cover a wide range of pathogenic MDRB strains such as EHEC and non-EHEC E. coli strains without producing remarkable bacterial resistance. According to the protocols and art used in the current invention, isolation of new 3-5 wild phages with full series of vertical optimization steps, plaque-based or biokinetic-based, does not take more than 2 weeks.

Phage Biocontrol, Bioprocessing and Animal Feed for Pathogenic E. coli

Despite the fact that a vast amount of work has been carried out on all aspects of E. coli since it was first described, the organism continues to provide new challenges to food safety. Although, in many countries including UK and USA, E. coli O157:H7 is currently the most predominant foodborne VTEC, it is not the only VTEC associated with foodborne illness: E. coli O26, O103, O111, O118 and O145 and other VTEC are causing significant morbidity in many countries and such serogroups are increasingly being recognized as posing an equal or possibly greater threat to human health than E. coli O157 (Bell and Kyriakids, 2002). Therefore, the design of this project was to create a reliable comprehensive phage cocktail which is highly capable for killing almost all serious pathogenic E. coli including E. coli serotype O157:H7. Given that, the previous efforts to contain E. coli spread was mistakenly focusing on only serotype O157 E. coli strains. This has lead to un-expected emergence of deadly epidemics by EPEC and ETEC and the discovered lately non-O157 EHEC strains.

It is the first time that such non-genetic breeding techniques are invented. Phage breeding was applied on E. coli which is Gram-negative bacteria that till now no satisfactory lysin extraction was succeeded. This imposes the importance of phage breeding along with phage hunting and phage optimization as the salvage for the historical setbacks of phage-therapy, bioprocessing, and biocontrol against pathogenic E. coli. On the other hand, the possibility for succeeding in separating phage lysins specific for E. coli now became closer because of the accessibility to much higher number of isolated and bred phages. Promising aspects of applying phage breeding techniques into other bacterial species became now possible especially for the multiple drug resistant (MDR) bacteria which are also resistant to phages lysis like Methicillin-resistant Staphylococcus aureus (MRSA), Mycobacterium tuberculosis and some strains of Salmonella.

Phage breeding could act as a non-perishable source of new lytic phages for E. coli, or any other bacterial species, therefore a new era of phage therapy, biocontrol and bioprocessing will start.

The bred phages via vertical or horizontal breeding techniques could be used effectively to treat one of the most money-consuming and health-endangering problems in the food and pharmaceutical and water industries, which is the bacterial biofilms including E. coli biofilms.

As most of the previously implemented phage-based diagnostic assays for bacteria were lacking the sufficient coverage of almost all strains of the targeted bacteria like E. coli, the current invention of phage design (hunting and breeding techniques) is being the solution. One of the great applications desired for the current invention is to formulate, for the first time, a highly reliable phage-based rapid diagnostic assay for detecting almost all pathogenic strains of E. coli including O157 E. coli serotypes in simple, sensitive, inexpensive and specific manner.

Possibility of Using the Current Invention (as a Principle) with Other Medically Important Bacteria

According to the current invention, it is possible to invest the breakthrough in the phage design for acquiring novel bred lytic phages against some of the most endangering MDR bacteria for example, but not limited to, MRSA, Pseudomonas aeruginosa, and Mycobacterium tuberculosis. The resulted phage master mix for each of the above listed dangerous bacteria will be able to be used in phage bio-processing, bio-control or fogging in hospitals and within the medical community, in the environment or in livestock (in case of MRSA) or even as topical phage therapy for MRSA or cutaneous Mycobacterium tuberculosis. It is possible to create a phage master mix for other food-borne pathogens like Salmonella, Staphylococcus aureus, Campylobacter jejuni, or to be used in food processing, or as preservatives or additives in food and beverages, or for water-borne pathogens such as Vibrio cholerae.

It could be used to manufacture phage-based rapid diagnostic tests for other bacteria rather than E. coli.

It could be used in preventing and/or treating biofilms formation on urinary catheters in hospital patients caused by other bacteria like Klebsiella, Proteus or Pseudomonas etc.

It could be used in the treatment of peptic ulcer and gastric/colorectal cancer inducing bacteria, namely Helicobacter pylori which is difficult to be eradicated by antibiotics. In this condition, it is needed to test the ability of phages to endure the low pH of the stomach or can be added with alkali base like sodium bicarbonate.

The phage master mix could be used to treat the “in side the body” bacterial biofilms, namely the bacterial adhesion and growth on the prosthetic components inside the body like heart valves, prosthetic joints etc. However, the main setback here is the development of immune reaction against the introduced phages.

### REFERENCES

Ackermann, H. W., and M. S. Dubow. 1987. Viruses of prokaryotes, vol. II. Natural groups of bacteriophages. CRC Press, Inc., Boca Raton, Fla.
Ahmed, R., Bopp, C., Borczyk, A. and Kasatiya, S. 1987. Phage-typing scheme for Escherichia coli O157:H7. J. Infect. Dis. 155, 806-809.
Alisky, J., Iczkowski, K., Rapoport, A., and Troitsky, N. 1998. Bacteriophages show promise as antimicrobial agents. J. Infect. 36:5-15.
Ausubel, F. M., Brent, R., Kingstone, R. E., Moore, D. D., Seidman, J. G., Smith, J. A. and Struhl, K. 1991. Growing lambda-derived vectors. In Current Protocols in Molecular Biology, Vol 1 ed. Asdf, A. pp. 12.1-12.3. New York: Wiley Interscience.
Barrangou, R, Fremaux, C, Deveau, H, Richards, M, Boyaval, P, Moineau, S, Romero, D A and Horvath, P. 2007. CRISPR provides acquired resistance against viruses in prokaryotes. Science 315: 1709-1712.
Barrow, P. A., and Soothill, J. S. 1997. Bacteriophage therapy and prophylaxis: rediscovery and renewed assessment of potential. Trends Genet. 5:268-271.
Barrow P, Lovell, M and Berchieri, A., Jr. 1998. Use of Lytic Bacteriophage for Control of Experimental Escherichia coli Septicemia and Meningitis in Chickens and Calves. Clinical and Diagnostic Laboratory Immunology, 5: 294-298.
Baxa, U.; Steinbacher, S.; Miller, S.; Weintraub, A.; Huber, R. and Seckler, R. 1996. Interactions of phage P22 tails with their cellular receptor, Salmonella O-antigen polysaccharide. Bioph. J., 71:2040-2048.
Bell, C., Kyriakids, A. 2002. Pathogenic Eshcerichia coli. In: Clive de W. Blackburn and Peter J. McClure (Eds.), Food borne pathogens Hazards, risk analysis and control. Woodhead publishing, England, pp 279-306.
Biswas, B., S. Adhya, P. Washart, B. Paul, A. N. Trostel, B. Powell, R. Carlton, and C. R. Merril. 2002. Bacteriophage therapy rescues mice bacteremic from a clinical isolate of vancomycin-resistant Enterococcus faecium. Infect. Immun. 70:204-210.
Bohannan, B. J. M., and R. E. Lenski. 1997. Effect of resource enrichment on a chemostat community of bacteria and bacteriophage. Ecology 78:2303-2315.
Brock, T. D. 1990. The emergence of bacterial genetics. Cold Spring Harbour Laboratory Press, Cold Spring Harbour, N.Y.
Calendar, R. (ed.). 1988. The bacteriophages, vol. 1. Plenum Press, New York, N.Y.
Carlton, R. M. 1999. Phage therapy: past history and future prospects. Arch. Immunol. Ther. Exp. 47:267-274.
Duenas, M and Borrebaeck, C A K. 1995. Novel helper phage design: intergenic region affects the assembly of bacteriophages and the size of antibody libraries. FEMS microbiology letters. 125: 317-321.
Edward, B and Ivana, I-B. 2007. Phenotypic effects of inactivating Cas-CRISPR genes in E. coli. In: Molecular genetics of bacteria and phages/Turnbough, Chuck; Dunny, Gary; Ades, Sarah (ed). —Madison, Wis., SAD: University of Wisconsin-Madison.
Garber, L., Wells, S., and Shroeder-tucker, L. 1999. Factors associated with the faecal shedding of verotoxin-producing Escherichia coli O157 on dairy farms. Journal of Food Protection. 62:307-12.
Goodridge, L., Chen, J and Griffiths. M W. 1999. Development and characterization of a fluorescent-bacteriophage assay for detection of Escherichia coli O157:H7. Applied and Environmental Microbiology 65: 1397-1404
Goodridge, L., Gallaccio, A. and Griffiths. M W. 2003. Morphological, host range, and genetic characterization of two coliphages. Applied and Environmental Microbiology 69:5364-5371
Heller, K. J. 1992. Molecular interaction between bacteriophage and the gram-negative cell envelope. Arch. Microbiol. 158:235-248.
Hoskisson, P A and Smith, M C M. 2007. Hypervariation and phase variation in the bacteriophage ‘resistome’. Current Opinion in Microbiology 10: 396-400.
Jackson, S. G., Goodbrand, R. B., and Johnson, R. P. 1998. Escherichia coli O157:H7 diarrhoea associated with well water and infected cattle on an Ontario farm. Epidemiology and Infection. 120: 17-20.
Jassim, S. A. A., Stewart, G. S. A. B. and Denyer, S. P. 1995. Selective virus culture. WO9523848A1.
Jassim, S A., A., Hibma, A. M. and Griffiths, M. W. 2005. The attachment efficiency of cell-walled and L-forms of Listeria monocytogenes to stainless steel. Journal of Food, Agriculture and Environment 3: 92-95.
Kudva, I T., Jelacic, S., Tarr, P I., Youderian, P. and Hovde, C J. 1999. Biocontrol of Escherichia coli O157 with O157-Specific Bacteriophages. Applied and Environmental Microbiology 65: 3767-3773.
Lenski, R. E., and B. R. Levin. 1985. Constraints on the coevolution of bacteria and virulent phage: a model, some experiments, and predictions for natural communities. Am. Nat. 125:585-602.
Letellier, L; Boulanger, P; Plançon, L; Jacquot, P; and Santamaria, M. 2004. Main features on tailed phage, host recognition and DNA uptake. Frontiers in Bioscience 9:1228-1339
Levin, B. R., F. M. Stewart, and L. Chao. 1977. Resource-limited growth, competition, and predation: a model and experimental studies with bacteria and bacteriophage. Am. Nat. 111:3-24.
Loretta, L. 1965. A nonspecific increase in permeability in Escherichia coli produced by EDTA. Journal of Microbiology. 53: 745-752.
Merril, C R, Scholl, D, Adhya, S L. 2003. The prospect for Bacteriophage therapy in Western medicine. Nat Rev Drug Discov; 2:489-497.
Merril, C, Biswis, B, Carlton, R, et al. 1996. Long-circulating bacteriophages as antibacterial agents. Proc Natl Acad Sci USA. 93:3188-3192.
Milne, L., Plom, M. A., and Strudley, I. 1999. Escherichia coli O157 incident associated with a farm open to members of the general public. Communicable Disease and Public Health. 2: 22-6.
Mizoguchi, K., M. Morita, C. Y. Fischer, M. Yoichi, Y. Tanji, and H. Unno. 2003. Coevolution of bacteriophage PP01 and Escherichia coli O157:H7 in continuous culture. Applied and Environmental Microbiology 69: 170-176.
Morita, M., Y. Tanji, K. Mizoguchi, T. Akitsu, N. Kijima, and H. Unno. 2002. Characterization of a virulent bacteriophage specific for Escherichia coli O157:H7 and analysis of its cellular receptor and two tail fibre genes. FEMS Microbiol. Lett. 211:77-83.
O'Sullivan, D., Coffey, A., Fitzgerald, G F., Hill, C., and Ross, R P. 1998. Design of a phage-insensitive lactococcal dairy starter via sequential transfer of naturally occurring conjugative pPlasmids. Appl Environ Microbiol. 64: 4618-4622.
Pelczar, M. J., Chan, E. C. S., Krieg, N. R., Edwards, D. D. and Pelczar, M. F. 1993. Viruses: Morphology, classification, replication. In Microbiology concepts and applications. Part VII, Chapter 15, pp. 401-435. McGraw-Hill, INC. New York.
Rieder, E., Berinstein, A., Baxt, B., Kangt, A. and Mason, P. W. 1996. Propagation of an attenuated virus by design: Engineering a novel receptor for a noninfectious foot-and-mouth disease virus. Proc. Natl. Acad. Sci. 93, 10428-10433.
Ronner, A. B. and Cliver, D. O. 1990. Isolation and characterization of a coliphage specific for Escherichia coli O157:H7. J. Food Protect. 53, 944-947.
Schade, S Z., Adler, J. and R is H. 1967. How bacteriophage chi attacks motile bacteria. J Virol 1: 599-609.
Steinbacher S, Miller S, Baxa U, Budisa N, Weintraub A, Seckler R and Huber R. 1997. Phage P22 tailspike protein: crystal structure of the head-binding domain at 2.3 A, fully refined structure of the endorhamnosidase at 1.56 A resolution, and the molecular basis of O-antigen recognition and cleavage. J Mol Biol. 267:865-880.
Stewart, G. S. A. B., Jassim, S. A. A., Denyer, S. P., Newby, P., Linley, K. and Dhir, V. K. 1998. The specific and sensitive detection of bacterial pathogens within 4 h using bacteriophage amplification. J Appl Bacteriol 84: 777-783.
Sulakvelidze, A., Alavidze, Z., and Morris, J. G. Jr. 2001. Bacteriophage therapy. Antimicrob. Agents Chemother. 45:649-659.
Tanji, Y, Shimada, T., Fukudomi, H., Miyanaga, K., Nakai, Y., Unno, H. 2005. Therapeutic use of phage cocktail for controlling Escherichia coli O157:H7 in gastrointestinal tract of mice. J Biosci Bioeng. 100:280-287.
Vieu J. F. 1975. Les Bacteriophages. In Fabre J. (ed.): Fraite de Therapeutique, Vol. Serums et Vaccins. Flammarion, Paris, 337-400.
Weber-Dabrowska, B, Mulczyk, M, Gorski, A. 2000. Bacteriophage therapy of bacterial infections: an update of our institute's experience. Arch Immunol Ther Exp. 48:547-551.

1-35. (canceled)

36. A method of modifying phage-host specificity, the method comprising incubating phages in a medium comprising of one or more of a chelating agent, detergent/surfactant, enzyme, lantibiotic, antibiotics and an agent which destroys cell walls.

37. A method according to claim 36, wherein the medium comprises one or more of EDTA, lysozyme, Nisin A and Tween® 20.

38. A method according to claim 36, wherein the medium comprises all of EDTA, lysozyme, Nisin A and Tween® 20.

39. A method according to claim 36, wherein the phage specificity is modified to infect previously resistant strains of the same bacteria.

40. A method according to claim 36, wherein the phage specificity is modified to infect different strains of bacteria.

41. A method according to claim 36, wherein the phage specificity is modified to infect a different species of bacteria.

42. A method of modifying phage-host specificity, the method comprising the steps of:—
(a) obtaining large amounts of wild-type phages from at least one natural source by incubating the phages with bacterial hosts to obtain large numbers of phages,
(b) removing bacterial host cells, to obtain a suspension of phages,
(c) plating the suspension of phages from step (b) on a lawn of bacterial host cells,
(d) assessing phage plaques to identify areas of highest phage activity,
(e) isolating the areas of highest phage activity and isolating phages therefrom,
(f) culturing the phages isolated in step (e) together with their host bacteria,
(g) adding a viricidal mixture to the culture media of step (f) to remove free phages from the culture medium,
(h) plating the viricidally-treated culture medium from step (g) onto a host bacterial lawn and identify plaques,
(i) removing the plaques showing most virulent phage activity from the plate and isolate the phages therefrom,
(j) incubating the phages obtained in step (i) in a medium comprising of one or more of a chelating agent, detergent/surfactant, enzyme, lantibiotic, antibiotics and an agent which destroys cell walls,
(k) isolating the bacteriophages of step (j) and incubating them in a growth medium,
(l) assessing the infectivity of the bacteriophages of step (k) and culturing those whose specificity has been modified,
(m) storing the bacteriophages cultured in step (l).

43. A method according to claim 42, wherein the bacteriophages are obtained from one or more of animal or bird faeces, animal or bird litter, plants, sewage, soil, or farmyard slurry.

44. A method according to claim 42, wherein the bacteriophages are obtained from one or a mixture of camel faeces, quellae litters, pigeons litters, chicken litters, sheep faeces, goat faeces, cattle faeces, cattle manure, cattle farms sewage and farm soil.

45. A method according to claim 42, wherein the bacteriophages are specific for one or more of Escherichia coli, Enterbacteriacea spp., Salmonella typhimurium, Pseudomonas aeruginosa, Bacterioides gingivalis, Actinobacillus actinomycetescomitans, Klebsiella pneumoniae, Gram positive bacteria, Staphylococcus aureus, MRSA, Streptococcus mutans, Listeria monocytogenes, Streptococcus agalactiae, Coryneform bacteria, Mycobacterium tuberculosis, Salmonella spp., Campylobacter jejuni, water-borne Vibrio cholerae, or Helicobacter pylori.

46. A method according to claim 42, wherein the bacteriophage infect one or more of Escherichia coli, Klebsiella pneumoniae, and Mycobacterium smegmatis.

47. A method according to claim 42, wherein steps (b) to (e) are carried out more than once.

48. A method according to claim 42, wherein steps (b) to (e) are carried out in the same reaction vessel.

49. A method of modifying phage-host specificity, the method comprising the steps of:—
(a) obtaining large amounts of wild-type phages from at least one natural source by incubating the phages with bacterial hosts to obtain large numbers of phages,
(b) removing bacterial host cells, to obtain a suspension of phages,
(c) plating the suspension of phages from step (b) on a lawn of bacterial host cells,
(d) assessing phage plaques to identify areas of highest phage activity,
(e) isolating the areas of highest phage activity and isolating phages therefrom,
(f) culturing the phages isolated in step (e) together with their host bacteria,
(g) adding a viricidal mixture to the culture media of step (f) to remove free phages from the culture medium,
(h) plating the viricidally-treated culture medium from step (g) onto a host bacterial lawn and identify plaques,
(i) removing the plaques showing most virulent phage activity from the plate and isolate the phages therefrom,
(j) incubating the phages obtained in step (i) in a medium comprising of one or more of a chelating agent, detergent/surfactant, enzyme, lantibiotic, antibiotics and an agent which destroys cell walls,
(k) isolating the bacteriophages of step (j) and incubating them in a growth medium,
(l) assessing the infectivity of the bacteriophages of step (k) and culturing those whose specificity has been modified,
(m) storing the bacteriophages cultured in step (l).
wherein step (d) further comprises the steps of assessing the biokinetics of the phage by
(i) taking a sample of phage from step (c) and adding it to a bacterial culture,
(ii) incubating the phage and bacteria together,
(iii) exposing the mixture of phage and bacteria to a viricidal agent, in the incubation vessel,
(iv) adding a surfactant the mixture in the incubation vessel and further incubating it, and
(v) adding culture broth to the incubation vessel and incubating prior to plating on a bacterial lawn and assessment of plaque morphology.

50. A method according to claim 49, wherein the phage and the bacteria are co-incubated prior to the addition of the viricidal agent for a period less than an hour.

51. A method according to claim 49, wherein the viricide comprises pomegranate rind extract, iron salts and a detergent or surfactant.

52. A method according to claim 49, wherein the viricide comprises pomegranate rind extract, iron salts and a detergent or surfactant.

53. A method according to claim 49, wherein the iron salt is ferrous sulphate (FeSO₄), and the detergent/surfactant is the polysorbate surfactant Tween® 20.

54. A phage produced by the method of claim 36.

55. A phage made by the method according to claim 42 in a preparation for the biocontrol of pathogenic E. coli in livestock, bioprocessing of machinery and tools, preservatives or additives in food or beverages, prevention of biofilm formation on medical or surgical devices including surgical implants, in phage-based rapid diagnostic testing, or in phage therapy for infection.
